SEQUENCE LISTING (87 sequences)

SEQ ID NO: 1 - DNA, 30 bp, Artificial Sequence
  primer
cgcggatcctgaactcctctggatctactg

SEQ ID NO: 2 - DNA, 30 bp, Artificial Sequence
  primer
ccgctcgagttacacggcgatctttccgcc

SEQ ID NO: 3 - DNA, 20 bp, Artificial Sequence
  primer
ctcggcctctgagctattcc

SEQ ID NO: 4 - DNA, 30 bp, Artificial Sequence
  primer
cgcggatccacaaccttcgcttcaaaaaat

SEQ ID NO: 5 - DNA, 24 bp, Artificial Sequence
  primer
gatcatagggataacagggtaatg

SEQ ID NO: 6 - DNA, 24 bp, Artificial Sequence
  primer
gatccattaccctgttatccctat

SEQ ID NO: 7 - DNA, 26 bp, Artificial Sequence
  primer
ggggatccatggattatcaagtgtca

SEQ ID NO: 8 - DNA, 26 bp, Artificial Sequence
  primer
ggggatcctcacaagcccacagatat

SEQ ID NO: 9 - DNA, 30 bp, Artificial Sequence
  primer
ccgctcgaggagccaagctctccatctagt

SEQ ID NO: 10 - DNA, 30 bp, Artificial Sequence
  primer
tacgaattcgatggattatcaagtgtcaag

SEQ ID NO: 11 - DNA, 30 bp, Artificial Sequence
  primer
cccaagcttgttctgggctcactatgctgc

SEQ ID NO: 12 - DNA, 30 bp, Artificial Sequence
  primer
agaggatccccgtatggaaaatgagagctg

SEQ ID NO: 13 - DNA, 30 bp, Artificial Sequence
  primer
tacgaattcgttaaagatagtcatcttggg

SEQ ID NO: 14 - DNA, 20 bp, Artificial Sequence
  primer
tcacaagcccacagatattt

SEQ ID NO: 15 - protein, 23 aa, Artificial Sequence
  CSNR1 zinc finger
YKCKQCGKAFGCPSNLRRHGRTH

SEQ ID NO: 16 - protein, 23 aa, Artificial Sequence
  DSAR2 zinc finger
YSCGICGKSFSDSSAKRRHCILH

SEQ ID NO: 17 - protein, 23 aa, Artificial Sequence
  DSCR zinc finger
YTCSDCGKAFRDKSCLNRHRRTH

SEQ ID NO: 18 - protein, 24 aa, Artificial Sequence
  DSNR zinc finger
YRCKYCDRSFSDSSNLQRHVRNIH

SEQ ID NO: 19 - protein, 23 aa, Artificial Sequence
  HSNK zinc finger
YKCKECGKAFNHSSNFNKHHRIH

SEQ ID NO: 20 - protein, 23 aa, Artificial Sequence
  HSSR zinc finger
FKCPVCGKAFRHSSSLVRHQRTH

SEQ ID NO: 21 - protein, 24 aa, Artificial Sequence
  ISNR zinc finger
YRCKYCDRSFSISSNLQRHVRNIH

SEQ ID NO: 22 - protein, 23 aa, Artificial Sequence
  KSNR zinc finger
YGCHLCGKAFSKSSNLRRHEMIH

SEQ ID NO: 23 - protein, 23 aa, Artificial Sequence
  QFNR zinc finger
YKCHQCGKAFIQSFNLRRHERTH

SEQ ID NO: 24 - protein, 23 aa, Artificial Sequence
  QNTQ zinc finger
YTCSYCGKSFTQSNTLKQHTRIH

SEQ ID NO: 25 - protein, 23 aa, Artificial Sequence
  QSHR2 zinc finger
YKCGQCGKFYSQVSHLTRHQKIH

SEQ ID NO: 26 - protein, 23 aa, Artificial Sequence
  QSHT zinc finger
YKCEECGKAFRQSSHLTTHKIIH

SEQ ID NO: 27 - protein, 23 aa, Artificial Sequence
  QSHV zinc finger
YECDHCGKSFSQSSHLNVHKRTH

SEQ ID NO: 28 - protein, 23 aa, Artificial Sequence
  QSNI zinc finger
YMCSECGRGFSQKSNLTIHQRTH

SEQ ID NO: 29 - protein, 23 aa, Artificial Sequence
  QSNK zinc finger
YKCEECGKAFTQSSNLTKHKKIH

SEQ ID NO: 30 - protein, 23 aa, Artificial Sequence
  QSNR1 zinc finger
FECKDCGKAFIQKSNLIRHQRTH

SEQ ID NO: 31 - protein, 23 aa, Artificial Sequence
  QSNV2 zinc finger
YVCSKCGKAFTQSSNLTVHQKIH

SEQ ID NO: 32 - protein, 23 aa, Artificial Sequence
  QSSR1 zinc finger
YKCPDCGKSFSQSSSLIRHQRTH

SEQ ID NO: 33 - protein, 23 aa, Artificial Sequence
  QSTR zinc finger
YKCEECGKAFNQSSTLTRHKIVH

SEQ ID NO: 34 - protein, 23 aa, Artificial Sequence
  QTHQ zinc finger
YECHDCGKSFRQSTHLTQHRRIH

SEQ ID NO: 35 - protein, 23 aa, Artificial Sequence
  QTHR1 zinc finger
YECHDCGKSFRQSTHLTRHRRIH

SEQ ID NO: 36 - protein, 25 aa, Artificial Sequence
  RDER1 zinc finger
YVCDVEGCTWKFARSDELNRHKKRH

SEQ ID NO: 37 - protein, 25 aa, Artificial Sequence
  RDER2
YHCDWDGCGWKFARSDELTRHYRKH

SEQ ID NO: 38 - protein, 24 aa, Artificial Sequence
  RDHR1 zinc finger
FLCQYCAQRFGRKDHLTRHMKKSH

SEQ ID NO: 39 - protein, 23 aa, Artificial Sequence
  RDHT zinc finger
FQCKTCQRKFSRSDHLKTHTRTH

SEQ ID NO: 40 - protein, 25 aa, Artificial Sequence
  RDKR zinc finger
YVCDVEGCTWKFARSDKLNRHKKRH

SEQ ID NO: 41 - protein, 23 aa, Artificial Sequence
  RSHR zinc finger
YKCMECGKAFNRRSHLTRHQRIH

SEQ ID NO: 42 - protein, 23 aa, Artificial Sequence
  RSNR zinc finger
YICRKCGRGFSRKSNLIRHQRTH

SEQ ID NO: 43 - protein, 23 aa, Artificial Sequence
  VDYK zinc finger
FHCGYCEKSFSVKDYLTKHIRTH

SEQ ID NO: 44 - protein, 23 aa, Artificial Sequence
  VSNV zinc finger
YECDHCGKAFSVSSNLNVHRRIH

SEQ ID NO: 45 - protein, 23 aa, Artificial Sequence
  VSSR zinc finger
YTCKQCGKAFSVSSSLRRHETTH

SEQ ID NO: 46 - protein, 23 aa, Artificial Sequence
  VSTR zinc finger
YECNYCGKTFSVSSTLIRHQRIH

SEQ ID NO: 47 - protein, 23 aa, Artificial Sequence
  WSNR zinc finger
YRCEECGKAFRWPSNLTRHKRIH

SEQ ID NO: 48 - protein, 23 aa, Artificial Sequence
  rdnq zinc finger
FACPECPKRFMRSDNLTQHIKTH

SEQ ID NO: 49 - protein, 23 aa, Artificial Sequence
  dghr zinc finger
YKCPECGKSFSDPGHLVRHQRTH

SEQ ID NO: 50 - protein, 23 aa, Artificial Sequence
  dgnv zinc finger
FQCRICMRNFSDSGNLRVHIRTH

SEQ ID NO: 51 - protein, 23 aa, Artificial Sequence
  sadr zinc finger
YKCPECGKSFSSPADLTRHQRTH

SEQ ID NO: 52 - protein, 23 aa, Artificial Sequence
  tldr zinc finger
YKCPECGKSFSTHLDLIRHQRTH

SEQ ID NO: 53 - protein, 23 aa, Artificial Sequence
  eshe zinc finger
YKCPECGKSFSERSHLREHQRTH

SEQ ID NO: 54 - protein, 23 aa, Artificial Sequence
  httn zinc finger
YKCPECGKSFSHRTTLTNHQRTH

SEQ ID NO: 55 - protein, 23 aa, Artificial Sequence
  rdev zinc finger
YKCPECGKSFSRRDELNVHQRTH

SEQ ID NO: 56 - protein, 23 aa, Artificial Sequence
  skae zinc finger
YKCPECGKSFSSKKALTEHQRTH

SEQ ID NO: 57 - protein, 23 aa, Artificial Sequence
  rdne zinc finger
YKCPECGKSFSRADNLTEHQRTH

SEQ ID NO: 58 - protein, 23 aa, Artificial Sequence
  tgne zinc finger
YKCPECGKSFSTSGNLTEHQRTH

SEQ ID NO: 59 - protein, 23 aa, Artificial Sequence
  thse zinc finger
YKCPECGKSFSTSHSLTEHQRTH

SEQ ID NO: 60 - protein, 23 aa, Artificial Sequence
  skhe zinc finger
YKCPECGKSFSSKKHLAEHQRTH

SEQ ID NO: 61 - protein, 23 aa, Artificial Sequence
  rdte zinc finger
YKCPECGKSFSRNDTLTEHQRTH

SEQ ID NO: 62 - protein, 23 aa, Artificial Sequence
  tnse zinc finger
YKCPECGKSFSTKNSLTEHQRTH

SEQ ID NO: 63 - protein, 23 aa, Artificial Sequence
  hghe zinc finger
YKCPECGKSFSHTGHLLEHQRTH

SEQ ID NO: 64 - protein, 23 aa, Artificial Sequence
  srta zinc finger
YKCPECGKSFSSRRTCRAHQRTH

SEQ ID NO: 65 - protein, 23 aa, Artificial Sequence
  qste zinc finger
YKCPECGKSFSQNSTLTEHQRTH

SEQ ID NO: 66 - protein, 23 aa, Artificial Sequence
  rdae zinc finger
YKCPECGKSFSRNDALTEHQRTH

SEQ ID NO: 67 - protein, 23 aa, Artificial Sequence
  tgae zinc finger
YKCPECGKSFSTTGALTEHQRTH

SEQ ID NO: 68 - protein, 23 aa, Artificial Sequence
  rdnt zinc finger
YKCPECGKSFSREDNLHTHQRTH

SEQ ID NO: 69 - protein, 339 aa, Artificial Sequence
  Z891R4
MVYPYDVPDYAELPPKKKRKVGIRIPGEKPYKCMECGKAFNRRSHLTRHQRIHTGEKPYRCKYCDRSFSISSNLQRHVRNIHTGEKPYRCKYCDRSFSISSNLQRHVRNIHTGEKPYTCSYCGKSFTQSNTLKQHTRIHTGEKQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 70 - protein, 337 aa, Artificial Sequence
  Z891F4
MVYPYDVPDYAELPPKKKRKVGIRIPGEKPFACPECPKRFMRSDNLTQHIKTHTGEKPYKCHQCGKAFIQSFNLRRHERTHTGEKPYKCMECGKAFNRRSHLTRHQRIHTGEKPYSCGICGKSFSDSSAKRRHCILHTGEKQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 71 - protein, 25 aa, Artificial Sequence
  yqrl zinc finger
HICHIQGCGKVYGQRSNLVRHLRWH

SEQ ID NO: 72 - protein, 25 aa, Artificial Sequence
  yrsl zinc finger
HICHIQGCGKVYGRSDALTRHLRWH

SEQ ID NO: 73 - protein, 25 aa, Artificial Sequence
  rtqn zinc finger
FMCTWSYCGKRFTQSGNLARHKRTH

SEQ ID NO: 74 - protein, 24 aa, Artificial Sequence
  tsgv zinc finger
MCTWSYCGKRFTTSGNLVRHKRTH

SEQ ID NO: 75 - protein, 23 aa, Artificial Sequence
  tanr zinc finger
FACPECPKRFMTSANLSRHIKTH

SEQ ID NO: 76 - protein, 23 aa, Artificial Sequence
  rdhr zinc finger
FACPECPKRFMRSDHLSRHIKTH

SEQ ID NO: 77 - protein, 23 aa, Artificial Sequence
  rdkr zinc finger
YKCPECGKSFSRSDKLVRHQRTH

SEQ ID NO: 78 - protein, 23 aa, Artificial Sequence
  qsnr zinc finger
YKCPECGKSFSQSSNLVRHQRTH

SEQ ID NO: 79 - protein, 23 aa, Artificial Sequence
  tgnr zinc finger
YKCPECGKSFSTSGNLVRHQRTH

SEQ ID NO: 80 - protein, 23 aa, Artificial Sequence
  dgnr zinc finger
YKCPECGKSFSDPGNLVRHQRTH

SEQ ID NO: 81 - protein, 23 aa, Artificial Sequence
  rder zinc finger
YKCPECGKSFSRSDELVRHQRTH

SEQ ID NO: 82 - protein, 23 aa, Artificial Sequence
  tger zinc finger
YKCPECGKSFSTSGELVRHQRTH

SEQ ID NO: 83 - protein, 23 aa, Artificial Sequence
  qaha zinc finger
YKCPECGKSFSQLAHLRAHQRTH

SEQ ID NO: 84 - protein, 23 aa, Artificial Sequence
  qssa zinc finger
YKCPECGKSFSQKSSLIAHQRTH

SEQ ID NO: 85 - protein, 23 aa, Artificial Sequence
  qgne zinc finger
YKCPECGKSFSQSGNLTEHQRTH

SEQ ID NO: 86 - protein, 23 aa, Artificial Sequence
  qgha zinc finger
YKCPECGKSFSQAGHLASHQRTH

SEQ ID NO: 87 - protein, 23 aa, Artificial Sequence
  rdht zinc finger
YKCPECGKSFSRSDHLTTHQRTH

(19) United States
(12) Patent Application Publication
(10) Pub. No.: US 2011/0281306 A1
(43) Pub. Date: Nov. 17, 2011

(54) Novel Zinc Finger Nuclease and Uses Thereof

(75) Inventors: Jin Soo Kim, Seoul (KR); Hye Joo Kim, Daejeon (KR)

(73) Assignee: TOOLGEN INCORPORATION, Seoul (KR)

(21) Appl. No.: 13/142,783

(22) PCT Filed: Sep. 18, 2009
(86) PCT No.: PCT/KR2009/005315

§ 371 Date: Aug. 4, 2011

(30) Foreign Application Priority Data
Dec. 31, 2008 (KR) 1020080138688

(51) Int. Cl. C12P 19/34; C07H 21/00; C12N 9/16

(52) U.S. Cl. 435/91.53; 435/196; 536/23.2

(57) ABSTRACT

The present invention relates to methods and compositions useful for targeted cleavage and alteration of a genomic sequence, targeted cleavage followed by homologous recombination between an exogenous polynucleotide and a genomic sequence, or targeted cleavage followed by non-homologous end joining.

### TECHNICAL FIELD

The present invention relates to methods and compositions useful for targeted cleavage and alteration of a genomic sequence, targeted cleavage followed by homologous recombination between an exogenous polynucleotide and a genomic sequence, or targeted cleavage followed by non-homologous end joining.

### BACKGROUND ART

Restriction enzymes are essential tools in genetic engineering and molecular biology. Various attempts have been made to create novel restriction enzymes known as “rare cutters” that recognize and cut specific DNA sequences of 9 or more base-pairs (bp). Of them, zinc finger nuclease technology functions to recognize and cut various DNA base sequences using a restriction enzyme which is a chimeric nuclease that consists of a zinc finger DNA-recognition domain and a DNA-cleavage domain. Zinc finger nuclease can be used for efficient genetic modifications of mammalian, plant, or other cells, since it is able to make a targeted double strand break (DSB) in the genomic DNA when introduced into cells. When a double strand break occurs in cells, the damaged region is repaired by the cell's own repair system. At this time, a donor DNA having a similar DNA sequence to the damaged region is introduced into the cells, leading to homologous recombination (HR) between the damaged DNA and donor template. For desired modification in a specific site of the genome, a target base sequence is incorporated into the donor DNA. Meanwhile, in the absence of a donor DNA, the damaged cells can be repaired by non-homologous end-joining (NHEJ). Non-homologous end-joining (NHEJ) repairs the damaged DNA by joining two broken ends together and usually produces no mutations. In some instances, however, the repair will be error-prone, resulting in insertion or deletion of base-pairs at the broken DNA ends. Taken together, a double strand break at a specific base sequence by zinc finger nuclease causes non-homologous end-joining, and thus the resulting DNA contains mutations, thereby producing knock-out cell lines.

Zinc fingers (ZFs), the most abundant DNA-binding motifs encoded in eukaryotic genomes, offer perhaps one of the best understood protein-DNA binding mechanisms (Wolfe, S. A. et al., (2000) Annu. Rev. Biophys. Biomol. Struct., 29, 183-212; Pabo, C. O. et al., (2001) Annu. Rev. Biochem., 70, 313-340; Moore, M. et al., (2003) Brief Funct. Genomic. Proteomic., 1, 342-355; and Klug, A. et al., (2005) FEBS Lett., 579, 892-894). Engineered zinc finger proteins (ZFPs) have significant potential as tools for gene regulation and genome modification because they can be used to target functional domains to virtually any desired location in any genome. Zinc finger proteins provide a versatile framework for designing proteins with new DNA-binding specificities, since they have the features of modular structure and base-specific interactions between bases and amino acid residues when recognizing the DNA base sequence.

The construction of effective zinc finger nucleases requires preparing zinc finger proteins that recognize desired target DNA sequences in the genome, which is recently achieved by various in vitro and in vivo selection methods, including replacement of each corresponding amino acid by site-directed mutagenesis (SMD) or a large scale screening method, phage display (see U.S. Pat. Nos. 5,789,538, 5,925,523, 6,007,988, 6,013,453, and 6,200,759; WO 95/19431, WO 96/06166, WO 98/53057, WO 98/54311, WO 00/27878, WO 01/60970, WO 01/88197, and WO 02/099084). However, these selection-based methods are highly labor-intensive and time-consuming, and require highly skilled technical staff. An alternative method for making ZFNs is to assemble pre-characterized single zinc finger modules into zinc finger arrays with a desired specificity by standard recombinant DNA technology. Each zinc finger module recognizes 3 by (base pairs) of DNA and, when appropriately joined together, the resulting zinc finger arrays are capable of specifically recognizing longer DNA sequence motifs. By using this method, zinc finger nucleases that recognize specific base sequences can be readily constructed. Some research groups have described and characterized separate archives of zinc finger modules for constructing multi-finger arrays. First, Barbas modules, developed using a combination of phage display and rational design methods by the Barbas laboratory at The Scripps Research Institute, are available for recognition of all GNN triplets, most ANN and CNN triplets, and a few TNN triplets. These Barbas modules were developed under the assumption that individual zinc finger modules have virtually complete positional independence, i.e. their recognition properties are not dramatically affected by their position within an array or by the identities of neighboring zinc fingers. Second, Sangamo modules, designed at Sangamo BioSciences Inc., are currently available for all GNN triplets and a smaller number of ‘non-GNN’ triplets. Sangamo modules were developed under the assumption that the position of a module within a 3-finger array can affect its recognition properties. Each of the three positions within a 3-finger array has a distinct zinc finger module developed for a given triplet at that position. However, since these modules are not naturally-occurring but artificial modules, they are not readily used for the construction of zinc finger nucleases, and have very low efficiencies in practical use. In particular, these modules are characterized in that they target GNN-repeat sequences. However, there is a problem in that these sequences occur only rarely in a given gene of interest. For example, a 3-finger ZFN pair can be designed to target the GNN-repeat DNA sequence, 5′-NNCNNCNNCNNNNNNGNNGNNGNN-3′, which occurs, on average, only once in a 4096 by (=46) sequence. GNN-repeat sites for 4-finger ZFNs are even more scarce, occurring, on average, only once in a 65,536 by (=48) sequence. Therefore, it is likely that such sites do not exist in many genes of interest. In this regard, the zinc finger nucleases of the present invention are advantageous in that most of them function without recognition of GNN-repeat sequences.

### DISCLOSURE OF INVENTION

### Technical Problem

As such, broad applications of zinc finger nuclease technology that allows targeted genome editing are hampered by the lack of a convenient, rapid and publicly available method for the synthesis of functional zinc finger nucleases. Thus, the present inventors have made an effort to develop a highly efficient and easy-to-practice modular-assembly method using publicly available zinc fingers to make zinc finger nucleases that are able to modify the DNA sequences of several different genomic sites in human cells. They found that the assembled zinc finger nucleases are more efficient tools for targeted cleavage and modification of the genome, compared to the known methods.

### Solution to Problem

It is an object of the present invention to provide a fusion protein, zinc finger nuclease comprising a zinc finger domain and a cleavage domain, in which the zinc finger domain includes three or more zinc finger modules, one or more of the zinc finger modules are derived from the naturally-occurring, wild-type zinc finger modules, and the fusion protein recognizes and cleaves a nucleotide sequence containing a region of interest in cellular chromatin.

It is another object of the present invention to provide a method for cleaving cellular chromatin in a region of interest using the zinc finger nuclease.

It is still another object of the present invention to provide a method for replacing a first nucleotide sequence in a region of interest in cellular chromatin using the zinc finger nuclease.

It is still another object of the present invention to provide a method for modifying a first nucleotide sequence in a region of interest in cellular chromatin using the zinc finger nuclease.

It is still another object of the present invention to provide a gene therapy method using the zinc finger nuclease.

### Advantageous Effects of Invention

The zinc finger nuclease of the present invention can be readily produced, and thus very useful for targeted cleavage and modification of a genomic sequence, which create gene knock-outs in many gene therapy applications.

### BRIEF DESCRIPTION OF DRAWINGS

FIG. 1 shows the source, amino acid sequence, and target subsite of zinc fingers used for the construction of zinc finger nucleases of the present invention.

FIG. 2 shows the amino acid sequence of a representative ZFN pair among the zinc finger nucleases synthesized by the present inventors; HA tag and nuclear localization signal sequences, which are included for the detection of their expression in the cell, are underlined, and the alpha helix regions of zinc fingers expected to bind with a target base sequence is denoted in bold.

FIG. 3 is a schematic overview of ZFN target sites in the CCR5 coding region; ZFN target sites are shown as boxes, the numbers indicate the positions of ZFN target sites relative to the start codon, and the crosshatched box indicates the locus of the 432 mutation.

FIG. 4 is a schematic illustration of the DNA cleavage assay, performed using ZFNs that were prepared by an in vitro transcription and translation system.

FIG. 5 is a photograph showing the results of DNA cleavage assay by agarose gel electrophoresis.

FIG. 6 is a schematic overview of cell-based single-strand annealing (SSA) for assessment of ZFN activities; ZFN expression plasmids are transfected into HEK293 cells whose genome contains an inactive, partially-duplicated and disrupted luciferase gene. If ZFNs cleave target sites in cells, DNA is efficiently repaired by the SSA mechanism and the functional luciferase gene is restored.

FIG. 7 is a schematic overview of cell-based single-strand annealing (SSA) for assessment of ZFN activities; Luciferase activities of cells indicate expression of various ZFNs. p3 is the empty plasmid, which was used as a negative control. The target sequence contains the recognition site of I-SceI, which was used as a positive control. The activity of each ZFN pair is reported as the percentage relative to the I-SceI control. ZFN pairs and their constituent monomers are indicated. The ZFN pairs used in further studies are marked with a triangle. Means and standard deviations (error bars) from at least three independent experiments are shown.

FIG. 8 is a schematic overview of mismatch detection of ZFN-mediated genome editing in human cells using T7E1 assay; Genomic DNA was purified from cells transfected with plasmids encoding ZFNs. The DNA segments encompassing the sites of ZFN recognition were PCR-amplified, and the DNA amplicons were melted and annealed. If the DNA amplicons contain both wild-type and mutated DNA sequences, heteroduplexes would be formed. T7E1 recognizes and cleaves heteroduplexes, but not homoduplexes. The DNA fragments were assessed by agarose gel electrophoresis.

FIG. 9 shows ZFN-mediated genomic modification revealed by the T7E1 assay; The ZFN pairs are shown at the top of the agarose gels. The expected positions of the resulting DNA bands are indicated by an arrow (uncut) and a bracket (cut) at the left of the gel panels. p3 is the empty plasmid used as a negative control. Sangamo's CCR5-targeting ZFN pair (Sangamo CCR5) was included in this assay.

FIG. 10 shows DNA sequences of a genomic site targeted by the Z836 ZFN pair; ZFN recognition elements are underlined. Deletions are indicated with dashes, and inserted bases are shown in small letters. In cases where a mutation was detected more than once, the number of occurrences is shown in parentheses. wt represents wild-type.

FIG. 11 shows DNA sequences of a genomic site targeted by the Z30 and Z266 ZFN pairs; ZFN recognition elements are underlined. Deletions are indicated with dashes, and inserted bases are shown in small letters. In cases where a mutation was detected more than once, the number of occurrences is shown in parentheses.

FIG. 12 shows DNA sequences of a genomic site targeted by the Z360 and Z411 ZFN pairs; ZFN recognition elements are underlined. Deletions are indicated with dashes, and inserted bases are shown in small letters. In cases where a mutation was detected more than once, the number of occurrences is shown in parentheses.

FIG. 13 shows DNA sequences of a genomic site targeted by the Z426 and Z430 ZFN pairs; ZFN recognition elements are underlined. Deletions are indicated with dashes, and inserted bases are shown in small letters. In cases where a mutation was detected more than once, the number of occurrences is shown in parentheses.

FIG. 14 shows DNA sequences of a genomic site targeted by the Z891 ZFN pair; ZFN recognition elements are underlined. Deletions are indicated with dashes, and inserted bases are shown in small letters. In cases where a mutation was detected more than once, the number of occurrences is shown in parentheses.

FIG. 15 shows types of mutations at various ZFN-targeted sites; The number of deletions, insertions and complex mutations for each ZFN pair were counted, and the percentages of these incidents were plotted.

FIG. 16 is the results of time course analysis of wild-type and obligatory heterodimeric ZFNs; The T7E1 mismatch detection assay was performed at various time points with DNA isolated from cells treated with different forms of the FokI nuclease domain. (WT, wild-type nuclease domain; RR/DD or KK/EL, obligatory heterodimeric nuclease domains)

FIG. 17 is the results of 53BP1 staining; Both the wild-type and obligatory heterodimeric Z891 ZFN pair were transfected into HEK293T/17 cells, and intracellular 53BP1 foci were detected by immunofluorescence at day 2 post-transfection. Etoposide (1 μM) was used as a positive control.

FIG. 18 is a plot of the result of FIG. 16; The distribution of the numbers of 53BP1 foci is plotted. At least 100 cells were analyzed for each treatment in two independent measurements.

FIG. 19 shows DNA sequences of mutant clones; 7 mutant clones were isolated, by limiting dilution, from cells treated with the RR/DD ZFN dimer. The DNA sequences of the target site in these clonal cells are shown. Deletions are indicated with dashes, and inserted bases are shown in small letters. Clone 1a and 1b indicate DNA sequences that resulted from biallelic modifications in a single clone.

FIG. 20 shows T7E1 assay at CCR2 sites; PCR-amplified DNA corresponding to the CCR2 coding region from cells treated with the ZFN pairs (shown at the top of the gel panels) was analyzed. ZFN pairs that gave rise to the modification at the CCR2 sites are marked by “+”. p3 is the empty plasmid used as a negative control. Sangamo's CCR5-targeting ZFN pair (Sangamo CCR5) was included in this assay.

FIG. 21 shows off-target effects of ZFNs at the CCR2 locus, and ZFN recognition elements at the CCR5 and CCR2 loci; The ZFN pairs are indicated at the left of the DNA sequences. The numbers of base matches between the CCR5 and CCR2 loci are indicated in parentheses, and mismatched bases are shown in lowercase bold letters. The half-site ZFN recognition elements are underlined.

FIG. 22 shows zinc fingers used for module swap experiments.

FIG. 23 is the results of the cell-based SSA system in module swap analysis to test whether these new ZFNs can functionally replace ZFNs of the present invention that recognize identical sequences; The ZFN monomers whose names start with “B” such as “BR4”, are composed exclusively of Barbas modules, and those with “S” such as “SR4”, are composed exclusively of Sangamo modules. Luciferase activities of cells indicate expression of various ZFNs. p3 is the empty plasmid, which was used as a negative control. The target sequence contains the recognition site of I-SceI, which was used as a positive control. The activity of each ZFN pair is reported as the percentage relative to the I-SceI control. ZFN pairs and their constituent monomers are indicated. Means and standard deviations (error bars) from at least three independent experiments are shown.

FIG. 24 shows ZFN-mediated genomic modification by the T7E1 assay in module swap analysis to test whether these new ZFNs can functionally replace ZFNs of the present invention that recognize identical sequences; ZFN pairs and their constituent monomers are shown at the top of the agarose gels. ZFN pairs that gave rise to positive gene targeting events are marked by “+”.

FIG. 25 is summary of ZFN analyses; The number of ZFN pairs or target sites that gave rise to positive results in each assay is shown.

FIG. 26 shows success rates of different types of ZFNs; Successful ZFN pairs or target sites are defined as those that gave rise to positive results in the T7E1 mismatch detection assay.

### BEST MODE FOR CARRYING OUT THE INVENTION

In accordance with one aspect, the present invention relates to a fusion protein, zinc finger nuclease (ZFN) comprising a zinc finger domain and a nucleotide cleavage domain, in which the zinc finger domain includes three or more zinc finger modules, two or more of the zinc finger modules are derived from the naturally-occurring, wild-type zinc finger modules, and the fusion protein recognizes and cleaves a nucleotide sequence containing a specific region in chromatin.

The zinc finger domain of the present invention refers to a protein that binds to a nucleotide in a sequence-specific manner through one or more zinc finger modules. The zinc finger domain includes at least three zinc finger modules. The zinc finger domain is often abbreviated as zinc finger protein or ZFP.

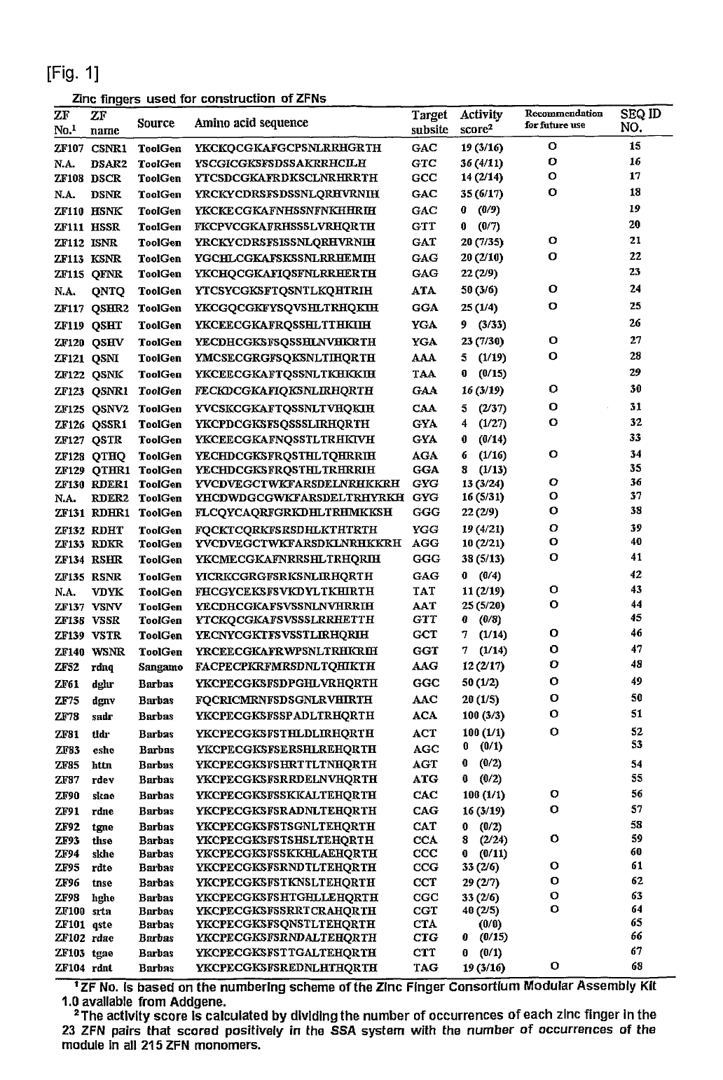

The zinc finger modules of the present invention are regions of amino acid sequence within the binding domain whose structure is stabilized through coordination of a zinc ion. The zinc finger modules of the present invention have the sequences being identical to those of the naturally-occurring, wild-type zinc finger modules or the sequences that are modified by substitution of other amino acids for any amino acids in the wild-type sequence. The wild-type zinc finger module may be derived from any eukaryotic cells, for example, fungal cells (e.g., yeast), plant or animal cells, (e.g., mammalian cells such as human or mouse). Preferably, the zinc finger module of the present invention includes the amino acid sequence, represented by FIG. 1, derived from human or the like.

The zinc finger domains of zinc finger nucleases consist of 3 or more tandemly arrayed zinc finger modules, each of which recognizes 3 by (base-pair) sub-sites. Since each module independently recognizes DNA sequences, the zinc finger domains consisting of 3 or 4 modules are able to bind to 9- or 12-bp sequence. Zinc finger nucleases function as dimmers and, therefore, a pair of zinc finger nuclease consisting of 3 or 4 modules specifically recognizes 18- to 24-bp sequence. In a specific example, the zinc finger nucleases of the present invention have the zinc finger domains consisting of 3 or 4 zinc finger modules, preferably 4 zinc finger modules. Further, in one preferred embodiment, the zinc finger domain contained in the zinc finger nuclease of the present invention preferably contains zinc finger modules. In the specific Example, 26% of potential cleavage sites were targeted successfully with zinc finger nucleases containing 4 zinc finger modules whereas only 9.1% of potential sites were targeted with zinc finger nucleases containing 3 zinc finger modules.

The zinc finger domains can be engineered to bind to a predetermined nucleotide sequence. Non-limiting examples of methods for engineering zinc finger domains are design and selection. A designed zinc finger protein is a protein not occurring in nature whose design/composition results principally from rational criteria. Rational criteria for design include application of substitution rules and computerized algorithms for processing information in database storing information of existing ZFP designs and binding data (see Korean patent No. 0766952). A selected zinc finger protein is a protein not found in nature whose production results primarily from an empirical process such as phage display, interaction trap or hybrid selection.

At least two or more of the zinc finger modules that constitute the processed zinc finger domains are preferably wild-type zinc finger modules, more preferably 3 or more wild-type zinc finger modules. In one specific Example, the present inventors performed module swap analysis in which the zinc finger nucleases of the present invention were replaced with those generated from zinc finger nucleases engineered and isolated by phage display or point mutation. It was found that the zinc finger nucleases of the present invention showed significant activity in the SSA system and T7E1 assay. Thus, it is preferable that the zinc finger nucleases of the present invention are composed of the naturally-occurring, wild-type zinc finger modules. More preferably, the zinc finger nucleases of the present invention are composed of zinc finger domains, represented by Table 2. In addition, the zinc finger nucleases of the present invention do not target GNN-repeat sequences, unlike the known engineered zinc finger nucleases. In the specific Example, among the 16 zinc finger nucleases that successfully modified the CCR5 sequences, only two (Z426F3 and Z360R4) recognized GNN-repeat sites, while two (Z430R3 and Z30F4) recognized half-site elements free of the GNN motif and 12 recognized sites that consisted of both GNN and non-GNN motifs, as shown in the following Table 2. The capability of targeting sites other than GNN-repeat sequences greatly expands the utility of ZFN technology.

As used herein, the term “cleavage” refers to the breakage of the covalent backbone of a DNA molecule, and the term “cleavage domain” refers to a polypeptide sequences which possesses catalytic activity for DNA cleavage.

The cleavage domain can be obtained from any endo- or exonuclease. Exemplary endonucleases from which a cleavage domain can be derived include, but are not limited to, restriction endonucleases and homing endonucleases. These enzymes can be used as a source of cleavage domains. In addition, both single-stranded cleavage and double-stranded cleavage are possible, in which double-stranded cleavage can occur depending on the source of cleavage domains. In this regard, the cleavage domain having double-strand cleavage activity may be used as a cleavage half-domain. Herein, the cleavage domain can be used interchangeably for single-stranded cleavage and double-stranded cleavage.

A cleavage domain can be derived from any nuclease or portion thereof. In general, two fusion proteins are required for cleavage if the fusion proteins comprise cleavage half-domains having double-strand cleavage activity. Two cleavage half-domains can be derived from the same endonuclease (or functional fragments thereof), or each cleavage half-domain can be derived from a different endonuclease (or functional fragments thereof). In addition, binding of two fusion proteins to their respective target sites places the cleavage half-domains in a spatial orientation to each other that allows the cleavage half-domains to form a functional cleavage domain, e.g., by dimerizing. Thus, any integral number of nucleotides or nucleotide pairs can intervene between two target sites (e.g., from 2 to 50 nucleotide pairs or more).

In general, if two fusion proteins are used, each comprising a cleavage half-domain, the primary contact strand for the zinc finger portion of each fusion protein will be on a different DNA strands and in opposite orientation. That is, for a pair of ZFP/cleavage half-domain fusions, the target sequences are on opposite strands and the two proteins bind in opposite orientations.

Restriction endonucleases are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme FokI catalyzes double-stranded cleavage of DNA, at 9 nucleotides from its recognition site on one strand and 13 nucleotides from its recognition site on the other. Thus, in one embodiment, fusion proteins comprise the cleavage domain (or cleavage half-domain) from at least one Type IIS restriction enzyme and one or more zinc finger binding domains.

Examples of the Type IIS restriction enzymes include FokI, AarI, AceIII, AciI, AloI, BaeI, Bbr7I, CdiI, CjePI, EciI, Esp3I, FinI, MboI, SapI, or SspD51, but are not limited thereto, more specifically, see Roberts et al. (2003) Nucleic acid Res. 31:418-420. In a specific embodiment, the FokI cleavage domain, obtained by separating a DNA binding domain from the Type IIS restriction enzyme FokI, was used as a cleavage domain (or half cleavage domain).

The fusion protein of the present invention refers to a polypeptide formed by the joining of two or more different polypeptides through a peptide bond. The polypeptides contain the zinc finger domain and nucleotide cleavage domain, which can cleave any target site in the nucleotide sequence. Herein, the fusion protein is used interchangeably with zinc finger nuclease or ZFN.

Methods for the design and construction of fusion proteins (or polynucleotide encoding fusion protein) are widely known in the art. In one specific embodiment, constructed was a polynucleotide that encodes a fusion protein containing a zinc finger domain and a cleavage domain. The polynucleotide may be inserted into a vector, and the vector may be introduced into a cell. In general, the components of the fusion proteins (e.g., ZFP-FokI fusion) are arranged such that the zinc finger domain is nearest the amino terminus (N-terminus) of the fusion protein, and the cleavage half-domain is nearest the carboxy-terminus (C-terminus). This mirrors the relative orientation of the cleavage domain in naturally-occurring dimerizing cleavage domains such as those derived from the FokI enzyme, in which the DNA-binding domain is nearest the amino terminus and the cleavage half-domain is nearest the carboxy terminus.

As used herein, the term “sequence” refers to a nucleotide sequence of any length, which can be DNA or RNA; can be linear, circular or branched and can be either single-stranded or double stranded.

As used herein, the term “binding” refers to a sequence-specific, or non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific. Such interactions are generally characterized by a dissociation constant (K_{d}) of 10⁻⁶M⁻¹ or lower. The term “affinity” refers to the strength of binding: increased binding affinity being correlated with a lower K_{d}.

In accordance with another aspect, the present invention relates to a recombination kit for cleavage, replacement or modification of DNA sequences in targeted region, comprising one or more pairs of zinc finger nucleases.

In general, because zinc finger nuclease (ZFNs) function as dimers, two ZFN monomers need to be prepared to target a single DNA site. Each of two monomeric ZFNs that compose a ZFN pair binds to one of the two 9- or 12-bp half-sites that are separated by a 5- or 6-bp spacer sequence. For a single half-site, multiple monomeric ZFNs can be designed, which consist of different sets of ZFs with identical or similar DNA-binding specificities. In the specific embodiment, ZFN pairs consisting of identical or similar zinc finger domains are shown in Table 2, but are not limited thereto. The single site can be targeted with many combinatorial ZFN pairs.

As used herein, the term “replacement” can be understood to represent replacement of one nucleotide sequence by another, (i.e., replacement of a sequence in the informational sense), and does not necessarily require physical or chemical replacement of one polynucleotide by another. As used herein, the term “modification” means a change in the DNA sequence by mutation or nonhomologous end joining. The mutation includes point mutations, substitutions, deletions, insertions or the like. The replacement or modification can replace or change a nucleotide having incomplete genetic information with a nucleotide having complete genetic information. The peptide encoded by the nucleotide sequence can be also functionally inactivated by the mutation. By this means, the zinc finger nuclease can be used as a tool for gene therapy. In one specific embodiment, it was confirmed that ZFNs targeted to knock out the human chemokine (C—C motif) receptor 5 (CCR5) gene, encoding the CCR5 protein that is the major co-receptor used by the human immunodeficiency virus (HIV) to infect target cells.

The term “recombinant” when used with reference, e.g., to a cell, nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (naturally occurring) form of the cell or express a second copy of a native gene that is otherwise normally or abnormally expressed, under expressed or not expressed at all.

In accordance with still another aspect, the present invention relates to a method for producing a zinc finger nuclease, the method comprising: (a) selecting a nucleotide sequence in the region of interest; (b) selecting zinc finger modules to bind to the sequence and engineering a zinc finger domain, in which the zinc finger domain includes three or more zinc finger modules, two or more of the zinc finger modules are derived from the naturally-occurring, wild-type zinc finger modules; and (c) expressing the fusion protein that comprises the engineered zinc finger domain and a nucleotide cleavage domain.

In the method for producing a zinc finger nuclease of the present invention, step (a) is a step of selecting a nucleotide sequence in the region of interest. The nucleotide sequence may be present inside or outside the cells, of the length is not limited. The nucleotide may be linear or circular, single-stranded or double-stranded.

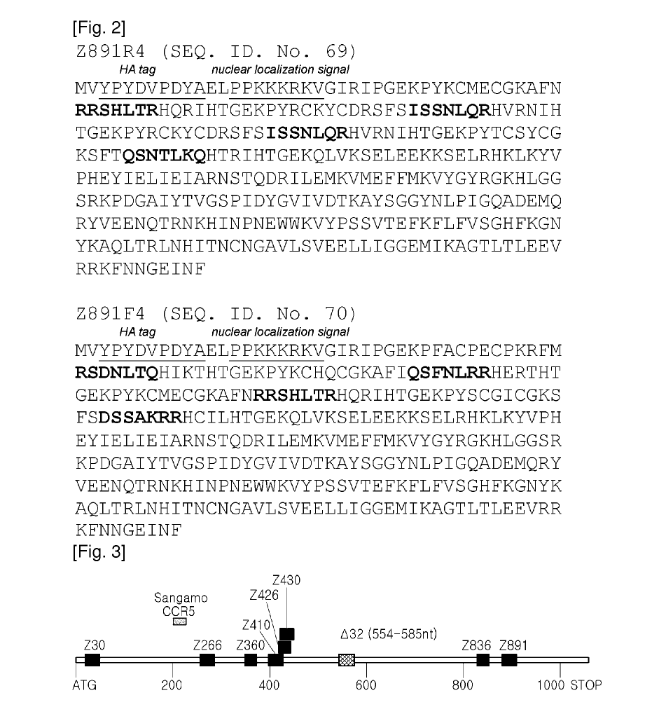

Step (b) is a step of selecting zinc finger modules to bind to a nucleotide sequence in the region of interest and engineering a zinc finger domain. Preferably, the zinc finger domain includes three or more zinc finger modules, two or more of the zinc finger modules are derived from the naturally-occurring, wild-type zinc finger modules, more preferably zinc finger modules represented by FIG. 1, and even more preferably zinc finger modules represented by FIG. 2.

Step (c) is a step of expressing the fusion protein that comprises the zinc finger domain and a nucleotide cleavage domain. The expression encompasses in vivo and ex vivo expression. In vivo expression may be performed by the method known in the art, for example, by using a vector. Examples of the vector include a plasmid, cosmid, bacteriophage and viral vector, but are not limited thereto. The suitable expression vector may be prepared by including secretory signal sequences as well as regulatory elements such as promoters, operators, initiation codons, termination codons, polyadenylation signals, and enhancers, depending on the purpose.

In accordance with still another aspect, the present invention relates to a method for cleaving cellular chromatin in a region of interest using the zinc finger nuclease.

In one specific embodiment, the present invention provides a method for cleaving cellular chromatin in a region of interest, comprising the steps of: (a) selecting a first nucleotide sequence in the region of interest; (b) selecting a first zinc finger nuclease of the present invention to bind to a first nucleotide sequence; (c) expressing the first zinc finger nuclease inside or outside the cells containing the nucleotide sequence, so as to introduce the nuclease into the cells; and (d) binding the first zinc finger nuclease (which is expressed in or introduced into the cells) to the sequence so as to cleave cellular chromatin in the region of interest.

The first zinc finger nuclease of the present invention includes three or more zinc finger modules, wherein two or more of the zinc finger modules are derived from naturally-occurring, wild-type zinc finger modules.

In another specific embodiment, the present invention provides a method for cleaving cellular chromatin in a region of interest, comprising the steps of: (a) selecting a first nucleotide sequence in the region of interest; (b) selecting a first zinc finger nuclease comprising a first zinc finger domain and a first nucleotide cleavage domain of the present invention to bind to a first nucleotide sequence; (c) expressing a first zinc finger nuclease inside or outside the cells containing the nucleotide sequence to introduce the nuclease into the cells; (d) binding a first zinc finger nuclease (which is expressed in or introduced into the cells) to the sequence so as to cleave cellular chromatin in the region of interest; (e) selecting and expressing a second zinc finger nuclease of the present invention inside or outside the cells containing the nucleotide sequence, so as to introduce the nuclease into the cells, wherein the second zinc finger nuclease comprises a second zinc finger domain and a second nucleotide cleavage domain; and (f) binding the second zinc finger nuclease (which is expressed in or introduced into the cells) to a second nucleotide sequence in the region of interest, wherein the second sequence is located between 2 and 50 nucleotides from the first nucleotide sequence to which the first zinc finger nuclease binds.

As used herein, “chromatin” refers to the nucleoprotein structure comprising the cellular genome. Cellular chromatin comprises nucleic acid, primarily DNA, and protein, including histones and non-histone chromosomal proteins. Cellular chromatin can be present in any type of cell, including prokaryotic and eukaryotic cells, such as fungal, plant, animal, mammalian, primate, and human cells. The majority of eukaryotic cellular chromatin exists in the form of nucleosomes, wherein a nucleosome core comprises approximately 150 base pairs of DNA associated with an octamer comprising two each of histones H2A, H2B, H3 and H4; and linker DNA (of variable length depending on the organism) extends between nucleosome cores. A molecule of histone H1 is generally associated with the linker DNA. For the purposes of the present disclosure, the term “chromatin” is meant to encompass all types of cellular nucleoprotein, both prokaryotic and eukaryotic. Cellular chromatin includes both chromosomal and episomal chromatin. A “chromosome” is a chromatin complex comprising all or a portion of the genome of a cell. An “episome” is a replicating nucleic acid, nucleoprotein complex or other structure comprising a nucleic acid that is not part of the chromosomal karyotype of a cell. Examples of episomes include plasmids and certain viral genomes.

In still another specific embodiment, the present invention provides a method for cleaving cellular chromatin in a region of interest, comprising the steps of: (a) selecting the region of interest; (b) providing a first zinc finger domain which binds to a first nucleotide sequence in the region of interest; (c) providing a second zinc finger domain which binds to a second nucleotide sequence in the region of interest, wherein the second sequence is located between 2 and 50 nucleotides from the first sequence; and (d) expressing the first zinc finger nuclease and the second zinc finger nuclease in the cells, simultaneously or separately, wherein the first zinc finger nuclease comprises a first zinc finger domain and a first nucleotide cleavage domain, and the second zinc finger nuclease comprises a second zinc finger domain and a second nucleotide cleavage domain;

wherein the first and second zinc finger domains include three or more zinc finger modules, two or more of the zinc finger modules are derived from naturally-occurring zinc finger modules, and

the first zinc finger domain binds to the first nucleotide sequence, and the second zinc finger domain binds to the second nucleotide sequence, and further wherein said binding positions the nucleotide cleavage domains such that the cellular chromatin is cleaved in the region of interest.

For targeted cleavage using the zinc finger nuclease of the present invention, the binding site of the zinc finger domain can encompass the cleavage site, or the near edge of the binding site can be between 1 and 50 nucleotides from the cleavage site. Methods for mapping cleavage sites are known to those of skill in the art.

The site at which the DNA is cleaved generally lies between the binding sites for the two fusion proteins. Double-strand breakage of DNA often results from two single-strand breaks, or “nicks”, offset by 1, 2, 3, 4, 5, 6 or more nucleotides, for example, cleavage of double-stranded DNA by native FokI results from single-strand breaks offset by 4 nucleotides. Thus, cleavage does not necessarily occur at exactly opposite sites on each DNA strand. In addition, the structure of the fusion proteins and the distance between the target sites can influence whether cleavage occurs adjacent a single nucleotide pair, or whether cleavage occurs at several sites.

As used herein, “target site” is the nucleic acid sequence recognized by a zinc finger domain. A single target site typically has about 6 to about 12 base pairs. Typically, a zinc finger protein having three zinc finger modules recognizes two adjacent 6 to 10 base-pair target sites, and a zinc finger protein having four zinc finger modules recognizes two adjacent 9 to 12 base-pair target sites. The term “adjacent target site” means a non-overlapping target site separated to 0 to about 5 base-pairs.

As noted above, the fusion protein can be introduced as polypeptides and/or polynucleotides. For example, two polynucleotides, each comprising sequences encoding one of the aforementioned polypeptides, can be introduced into a cell, and cleavage occurs at or near the target sequence. Alternatively, a single polynucleotide comprising sequences encoding both polypeptides is introduced into a cell. Polynucleotides can be DNA, RNA, or any modified forms or analogues of DNA and/or RNA.

In accordance with still another aspect, the present invention relates to a method for replacing a first nucleotide sequence in a region of interest in cellular chromatin using the zinc finger nuclease.

In a specific embodiment, the present invention provides a method for replacing a first nucleotide sequence in a region of interest in cellular chromatin, comprising the steps of: (a) engineering a first zinc finger domain to bind to a second nucleotide sequence in the region of interest, wherein the second sequence comprises at least 9 nucleotides; (b) providing a second zinc finger domain to bind to a third nucleotide sequence, wherein the third sequence comprises at least 9 nucleotides and is located between 2 and 50 nucleotides from the second sequence; (c) expressing a first zinc finger nuclease and a second zinc finger nuclease in the cells, wherein the first zinc finger nuclease comprises a first zinc finger domain and a second nucleotide cleavage domain, and the second zinc finger nuclease comprises a second zinc finger domain and a third nucleotide cleavage domain; and (d) exposing the cell to a polynucleotide comprising a fourth nucleotide sequence, wherein the fourth nucleotide sequence is homologous but non-identical with the first nucleotide sequence;

wherein the first and second zinc finger domains include three or more zinc finger modules, one or more of the zinc finger modules are derived from naturally-occurring zinc finger modules, and

binding of the first zinc finger nuclease to the second sequence, and binding of the second zinc finger nuclease to the third sequence, positions the cleavage domains such that the cellular chromatin is cleaved in the region of interest, thereby facilitating homologous recombination between the first nucleotide sequence and the fourth nucleotide sequence, resulting in replacement of the first nucleotide sequence with the fourth nucleotide sequence.

The present disclosure provides methods of targeted sequence alteration characterized by a greater efficiency of targeted recombination and a lower frequency of non-specific insertion events. Because double-stranded breaks in cellular DNA stimulate homologous recombination several thousand-fold in the vicinity of the cleavage site, such targeted cleavage allows for the alteration or replacement (via homologous recombination) of sequences at virtually any site in the genome.

In addition to the fusion protein (zinc finger nuclease), targeted replacement of a selected genomic sequence also requires a donor sequence. The donor sequence can be introduced into the cell prior to, concurrently with, or subsequent to, expression of the fusion protein(s). It will be readily apparent that the donor sequence is typically not identical to the genomic sequence that it replaces. For example, the sequence of the donor polynucleotide can contain one or more single base changes, insertions, deletions, inversions or rearrangements with respect to the genomic sequence, so long as sufficient homology is present to support homologous recombination. Alternatively, a donor sequence can contain a non-homologous sequence flanked by two regions of homology. Additionally, donor sequences can comprise sequences that are not homologous to the region of interest in cellular chromatin.

Further, in the method, the first nucleotide sequence may be preferably a sequence comprising a mutation in the gene. In this case, the donor sequence, in particular, the fourth sequence is preferably a wild-type sequence of the gene. The mutation may include point mutations, substitutions, deletions or insertions.

Further, in still another specific embodiment, the present invention provides a method for altering a first nucleotide sequence in a region of interest in cellular chromatin using the zinc finger nuclease.

Specifically, the present invention provides a method for altering a first nucleotide sequence in a region of interest in cellular chromatin, comprising the steps of: (a) engineering a first zinc finger domain to bind to a second nucleotide sequence in the region of interest, wherein the second sequence comprises at least 9 nucleotides; (b) providing a second zinc finger domain to bind to a third nucleotide sequence, wherein the third sequence comprises at least 9 nucleotides and is located between 2 and 50 nucleotides from the second sequence; and (c) expressing a first zinc finger nuclease and a second zinc finger nuclease in the cells, wherein the first zinc finger nuclease comprises a first zinc finger domain and a second nucleotide cleavage domain, and the second zinc finger nuclease comprises a second zinc finger domain and a third nucleotide cleavage domain; wherein the first and second zinc finger domains include three or more zinc finger modules, one or more of the zinc finger modules are derived from the naturally-occurring, wild-type zinc finger modules, and binding of the first zinc finger nuclease to the second sequence, and binding of the second zinc finger nuclease to the third sequence, positions the cleavage domains such that the cellular chromatin is cleaved in the region of interest, resulting in alteration of the first nucleotide sequence at the cleavage site.

In the present invention, the nucleic acids encoding one or more ZFPs or ZFP fusion proteins may be typically cloned into vectors for transformation into prokaryotic or eukaryotic cells for replication and expression. Vectors are typically prokaryotic or eukaryotic vectors, e.g., plasmids, shuttle vectors, or insect vectors. The nucleic acid encoding a ZFP is also typically cloned into an expression vector, for administration to a plant cell, animal cell, preferably a mammalian cell or a human cell, fungal cell, bacterial cell, or protozoal cell.

An expression vector is a nucleic acid construct, generated recombinantly or synthetically, with a series of specified nucleic acid elements that permit transcription of a particular nucleic acid in a host cell, and optionally integration or replication of the expression vector in a host cell. The expression vector can be part of a plasmid, virus, or nucleic acid fragment, of viral or non-viral origin. Typically, the expression vector includes an “expression cassette”, which comprises a nucleic acid to be transcribed operably linked to a promoter. The term expression vector also encompasses naked DNA operably linked to a promoter.

To obtain expression of a cloned gene or nucleic acid, the sequence encoding a ZFP or ZFP fusion protein is typically subcloned into an expression vector that contains a promoter to direct transcription. Suitable bacterial and prokaryotic promoters are well known in the art.

The terms “operably linked” refers to functional linkage between a nucleic acid expression control sequence (such as a promoter or array of transcription factor binding sites) and a second nucleic acid sequence.

In the present invention, “host cell” means a cell that contains zinc finger nuclease, or an expression vector or nucleic acid, either of which optionally encodes the zinc finger nuclease. The host cell typically supports the replication or expression of the expression vector. Host cells can be prokaryotic cells such as E. coli, or eukaryotic cells such as yeast, fungal, protozoal, higher plant, and insect cells, or amphibian cells, or mammalian cells such as CHO, HeLa 293, COS-1, and HEK, e.g., cultured cells (in vitro), explants and primary cultures (in vitro and ex vivo), and cells in vivo.

The term “nucleic acid” refers to deoxyribonucleotides or ribonucleotides and polymers thereof in single- or double-stranded form. The term encompasses nucleic acids containing known nucleotide analogs or modified backbone residues or linkages, which are synthetic, naturally occurring, and non-naturally occurring, which have similar binding properties as the reference nucleic acid. Examples of such analogs include, without limitation, phosphorothioates, phosphoramidates, methyl phosphonates, chiral-methyl phosphonates, 2-O-methyl ribonucleotides, peptide-nucleic acids (PNAs).

As used herein, the terms “polypeptide”, “peptide”, and “protein” are used interchangeably to refer to a polymer of amino acid residues. The term also applies to amino acid polymers in which one or more amino acid residues is an artificial chemical mimetic of a corresponding naturally occurring amino acid, as well as to naturally occurring amino acid polymers and non-naturally occurring amino acid polymers.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

Hereinafter, the present invention will be described in more detail with reference to Examples. However, these Examples are for illustrative purposes only, and the invention is not intended to be limited by these Examples.

### MODE FOR THE INVENTION

### EXAMPLE 1

### ZFN Design and Construction of ZFN-Containing Plasmid

DNA segments that encoded 3- or 4-finger arrays were assembled as described in “Human zinc fingers as building blocks in the construction of artificial transcription factor” (Bae, K. H. et al. Nat Biotechnol 21, 275-280 (2003)). Of the 3- or 4-finger arrays, 54 zinc finger modules (hereinbelow, referred to as ZF module) with diverse DNA-binding specificities were chosen. The selected ZF modules and amino acid sequences thereof are shown in FIGS. 1 and 2. Of these 54 ZF modules, 31 were derived from DNA sequences in the human genome and 2 were from those in the Drosophila genome (FIG. 1). The ZF modules derived from DNA sequences in the human and Drosophila genomes are termed ToolGen modules, for the sake of convenience. The remaining 21 ZF modules were either selected from libraries of ZF variants using phage display (Barbas modules) or produced by site-directed mutagenesis (Sangamo modules).

Next, for the design of multi-finger ZFNs, we used a computer algorithm that was developed at ToolGen to identify potential ZFN target sites in the DNA sequence of the human chemokine (C−C motif) receptor 5 (CCR5) coding region. On the basis of the results, the present inventors synthesized 208 ZFN monomers in combinations of 54 ZF modules, and the amino acid sequence of a representative ZFN pair is shown in FIG. 2. Collectively, these 315 ZFN pairs were intended to target 33 sites in the CCR5 gene (see FIG. 3).

The zinc finger domains that were composed of 3 or 4 ZF modules were integrated into the FokI endonuclease domain derived from flavobacterium okeanokoites genomic DNA, and the integrated sequence was cloned into p3, which is a modified version of the pcDNA3.0 plasmid (Invitrogen).

### EXAMPLE 2

### In vitro and in vivo Assays of ZFN Activity

2-1. In vitro Digestion Assay

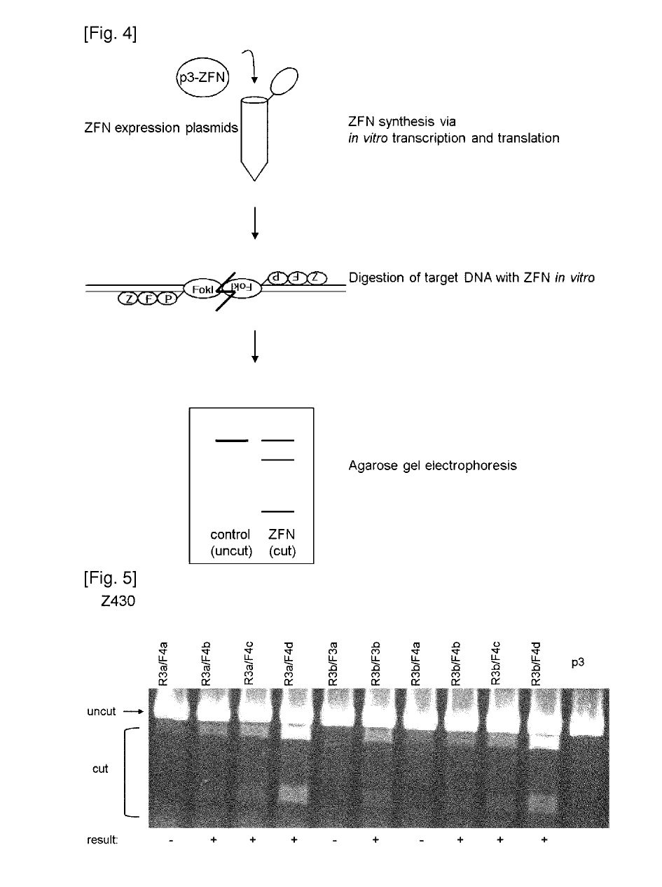

First, ZFNs were prepared using an in vitro transcription and translation (IVTT) system and incubated with a DNA segment that contained the CCR5 coding sequence to assess their DNA restriction activities in vitro. Specifically, the designed ZFNs in Example 1 were transcribed and translated in vitro using the TnT-Quick coupled transcription/translation system (Promega) as described by the manufacturer (in vitro transcription and translation; IVTT). Briefly, plasmids (0.5 μl each) that encoded ZFNs were added to TnT Quick master mix (20 μl) and 1 mM methionine (0.5 μl), and incubated for 90 min at 30° C. The target plasmid containing the CCR5 coding sequence was first digested with the restriction enzyme and made linear. The target plasmid (1 μg) was then digested by incubation with a pair of ZFN IVTT lysates (1 μl each) for 2 hrs at 37° C. in NEBuffer 4 (New England Biolabs). The reaction mixtures were inactivated by heat (65° C.) and then centrifuged at 13,000 rpm. The supernatant was used for agarose gel analysis. FIG. 4 is schematic illustration of the assay, and the results are shown in FIG. 5.

As shown in FIG. 5, many ZFN pairs (40%) showed efficient site-specific cleavage of the target DNA.

2-2: Single-Strand Annealing System

The present inventors then tested whether these ZFNs were able to induce homologous recombination in human cells using a mammalian cell-based single-strand annealing (SSA) system.

2-2-1. Reporter Plasmid Used in Cell-Based Assays

The 3′portion of the firefly luciferase gene was amplified from pGL3-control (Promega) using the primers 1 and 2 of the following Table 1, and the amplified product was cloned into the BamHI and XhoI sites of pcDNA5/FRT/TO (Invitrogen). The 5′portion of the luciferase gene amplified using the primers 3 and 4 of the following Table 1 was sequentially cloned into the HindIII and BamHI site of the resulting plasmid. I-SceI binding site was cloned into the BamHI site of the reporter plasmid using primers 5 and 6 of the following Table 1. The CCR5 coding sequence amplified using primers 7 and 8 was cloned into the BamHI site of the reporter plasmid.

TABLE 1
[Table 1]

| Primer | | |
|---|---|---|
| number | Sequence (5′ to 3′) | SEQ ID NO. |
| 1 | CGCGGATCCTGAACTCCTCTGGATCTACTG | SEQ ID NO. 1 |
| 2 | CCGCTCGAGTTACACGGCGATCTTTCCGCC | SEQ ID NO. 2 |
| 3 | CTCGGCCTCTGAGCTATTCC | SEQ ID NO. 3 |
| 4 | CGCGGATCCACAACCTTCGCTTCAAAAAAT | SEQ ID NO. 4 |
| 5 | GATCATAGGGATAACAGGGTAATG | SEQ ID NO. 5 |
| 6 | GATCCATTACCCTGTTATCCCTAT | SEQ I0 NO. 6 |
| 7 | GGGGATCCATGGATTATCAAGTGTCA | SEQ ID NO. 7 |
| 8 | GGGGATCCTCACAAGCCCACAGATAT | SEQ ID NO. 8 |
| 9 | CCGCTCGAGGAGCCAAGCTCTCCATCTAGT | SEQ ID NO. 9 |
| 10 | TACGAATTCGATGGATTATCAAGTGTCAAG | SEQ ID NO. 10 |
| 11 | CCCAAGCTTGTTCTGGGCTCACTATGCTGC | SEQ ID NO. 11 |
| 12 | AGAGGATCCCCGTATGGAAAATGAGAGCTG | SEQ ID NO. 12 |
| 13 | TACGAATTCGTTAAAGATAGTCATCTTGGG | SEQ ID NO. 13 |
| 14 | TCACAAGCCCACAGATATTT | SEQ ID NO. 14 |

2-2-2. Cell Culture and Establishment of Reporter Cell Line

HEK293T/17 (ATCC, CRL-11268™) cells and Flp-In™ T-REx™ 293 cells (Invitrogen), transformed with the plasmid containing the ZFN coding sequence in Example 1, were maintained in Dulbecco's modified Eagle medium (DMEM) supplemented with 100 units/ml penicillin, 100 μg/ml streptomycin, 0.1 mM non-essential amino acids and 10% fetal bovine serum (FBS). The reporter plasmid encoding the disrupted luciferase gene was stably integrated into Flp-In™ T-Rex™ 293 cells according to the manufacturer's instruction. Briefly, the cells were co-transfected with the reporter plasmid and pOG44, the Flp recombinase expression vector, and selected using hygromycin B. A clonal cell line bearing the disrupted luciferase gene was identified and used for the SSA assay.

2-2-3. Cell-Based Assay Using the SSA System

Each pair of ZFN expression plasmids (100 ng each) was transfected into 30,000 reporter cells/well in a 96-well plate format using Lipofectamine 2000 (Invitrogen). After 48 hrs, the luciferase gene was induced by incubation with doxycycline (1 μg/ml). After 24 hrs of incubation, the cells were lysed in 20 μl of 1× lysis buffer (Promega), and luciferase activity was determined using 10 μl of luciferase assay reagent (Promega) plus 2 μl of cell lysate.

2-2-4. Results

Plasmids that encoded ZFNs were transfected into human embryonic kidney cells

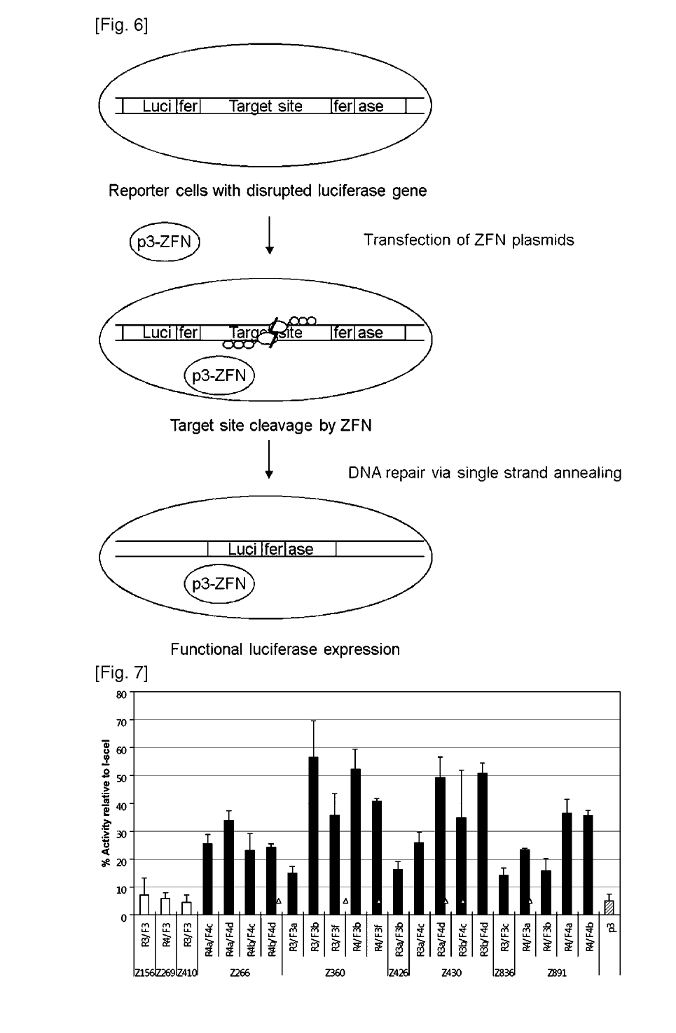

(HEK cells) whose genome contained a stably-integrated, partially-duplicated firefly luciferase gene that was disrupted by insertion of the CCR5 sequence. Effective ZFNs would generate a double strand break (DSB) in the CCR5 sequence, which should allow the functional luciferase gene to be restored via SSA. The efficiency of DNA cleavage by the ZFNs can be estimated by measuring luciferase enzyme activity. The highly efficient meganuclease, I-SceI was used as a positive control. Schematic overview of the experimental procedure is shown in FIG. 6 and the results are shown in FIG. 7.

As shown in FIG. 7, many ZFN pairs yielded significant luciferase activity in this assay. Out of 315 ZFN pairs, 23 pairs showed 15 to 57% luciferase activity, compared with the positive control I-SceI. It is possible that ZFN pairs that exhibited less than 15% activity still would be able to induce double strand break in cells, but the present inventors focused on the highly active 23 pairs for further studies. These 23 ZFN dimers did not yield significant luciferase activity when they used target-less reporter cells, whose genome contained the partially duplicated luciferase gene disrupted by a DNA sequence unrelated to CCR5 (data not shown). These results suggest that ZFN-mediated DNA repair is CCR5-specific.

### EXAMPLE 3

### Mutation Detection in HEK293 Cells Treated with ZFNs

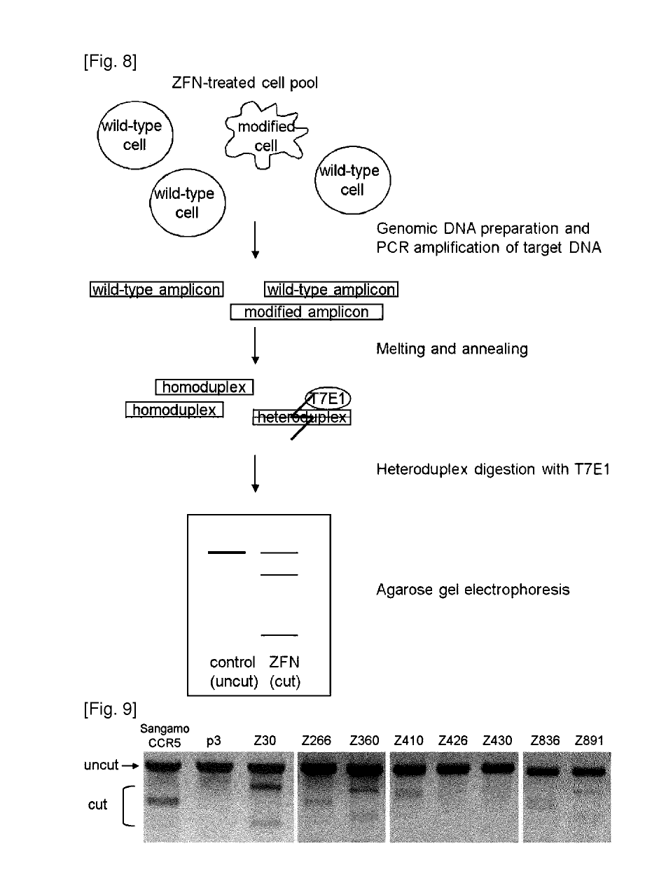

The present inventors investigated whether the ZFNs that both displayed endonuclease activity in the IVTT assay and induced luciferase activity in the cell-based assay could modify endogenous target sequences in cells. A double strand break induced by ZFNs can be repaired by error-prone non-homologous end joining (NHEJ). The resulting DNA often contains small insertion or deletions (indel mutations) near the DSB site. These indel mutations can be detected in vitro by treating amplified DNA fragments with mismatch-sensitive T7 endonuclease I (T7E1) (see FIG. 8).

Specifically, HEK293T/17 cells pre-cultured in a 96-well plate were transfected with two plasmids encoding a ZFN pair (100 ng each) using Lipofectamine 2000 (Invitrogen). After 72 hrs of incubation, the genomic DNA was extracted from ZFN-transfected cells using G-spin™ Genomic DNA extraction kit (iNtRON BIOTECHNOLOGY) as described by the manufacturer. The genomic region encompassing the ZFN target site was amplified, melted and annealed to form heteroduplex DNA. The primers 9 and 12 were used for the Z30 site, 10 and 12 for the Z266 and Z360 sites, 11 and 12 for the Z410, Z426, and Z430 sites, and 13 and 14 for the Z836 and Z891 sites (see Table 1). The annealed DNA was treated with 5 units of T7 endonuclease 1 (New England BioLabs) for 15 min at 37° C. and then precipitated by addition of 2.5 volumes of ethanol. The precipitated DNA was analyzed by agarose gel electrophoresis. The results are shown in FIG. 9.

As shown in FIG. 9, small fractions of amplified DNA from ZFN-treated cells were cleaved by T7E1. Each ZFN pair gave rise to distinctive cleavage patterns, reflecting the fact that these ZFNs targeted 8 different sites. In addition, the sizes of the resulting DNA bands were as expected. Out of 23 ZFN pairs tested in the T7E1 assay, 21 showed detectable DNA bands with expected size (data not shown).

The present inventors also used the T7E1 assay to test representative ZFN pairs that either did not show significant endonuclease activity in the IVTT assay or did not give rise to significant luciferase activity in the cell-based SSA system, but passed the IVTT test. The T7E1 assay results showed that none of these ZFN pairs induced detectable levels of DNA mutation.

### EXAMPLE 4

### DNA Sequence Analyses of Mutations Induced by ZFNs

The present inventors chose for further study 8 representative ZFN pairs, each of which targeted different sites in the CCR5 gene (see Table 2). They first examined whether any of these 8 individual ZFNs can function as homo-dimers. The gene targeting activity of each of the ZFN monomers alone was analyzed using the T7E1 assay. As expected, none of the ZFN monomers were able to generate detectable DNA cleavage (data not shown).

TABLE 2
[Table 2]

| | | | | | | Number |
|---|---|---|---|---|---|---|
| | | | | | Half-site | of |
| ZFN | | | | | sequence | GNN |
| name | F1 | F2 | F3 | F4 | (5′ to 3′) | motifs |
| Z30R4 | tldr | RDHT | ISNR | QNTQ | ATA GAT TGG ACT | 1 |
| Z30F4 | sadr | VDYK | VDYK | VSNV | AAT TAT TAT ACA | 0 |
| Z266R4 | rdne | DSCR | QSHV | rdnt | TAG TGA GCC CAG | 1 |
| Z266F4 | QSHR2 | RDER2 | thse | hghe | CGC CCA GTG GGA | 2 |
| Z360R4 | QSNR1 | QSNR1 | ISNR | ISNR | GAT GAT GAA GAA | 4 |
| Z360F3 | QSHV | VSNV | DSNR | | GAC AAT CGA | 2 |
| Z410R4 | sadr | dgnv | QSSR1 | QSNI | AAA GCA AAC ACA | 1 |
| Z410F4 | skae | WSNR | CSNR1 | rdne | CAG GAC GGT CAC | 2 |
| Z426R3 | tnse | srta | DSNR | | GAC CGT CCT | 1 |
| Z426F3 | RDER2 | RDER1 | RDHR1 | | GGG GTG GTG | 3 |
| Z430R3 | rdte | QSHV | RDKR | | AGG TGA CCG | 0 |
| Z430F4 | RDER2 | QSNV2 | QSHV | RDHT | TGG TGA CAA GTG | 1 |
| Z836R3 | RDHT | VSTR | QNTQ | | ATA GCT TGG | 1 |
| Z836F3 | CSNR1 | QTHQ | DSNR | | GAC AGA GAC | 2 |
| Z891R4 | RSHR | ISNR | ISNR | QNTQ | ATA GAT GAT GGG | 3 |
| Z891F4 | rdnq | QFNR | RSHR | DSAR2 | GTC GGG GAG AAG | 3 |

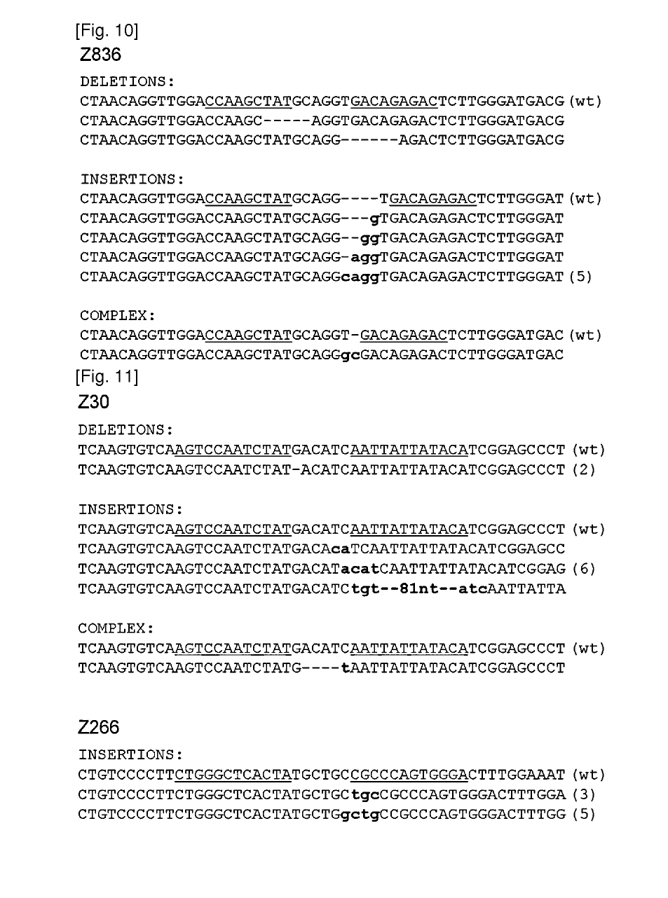

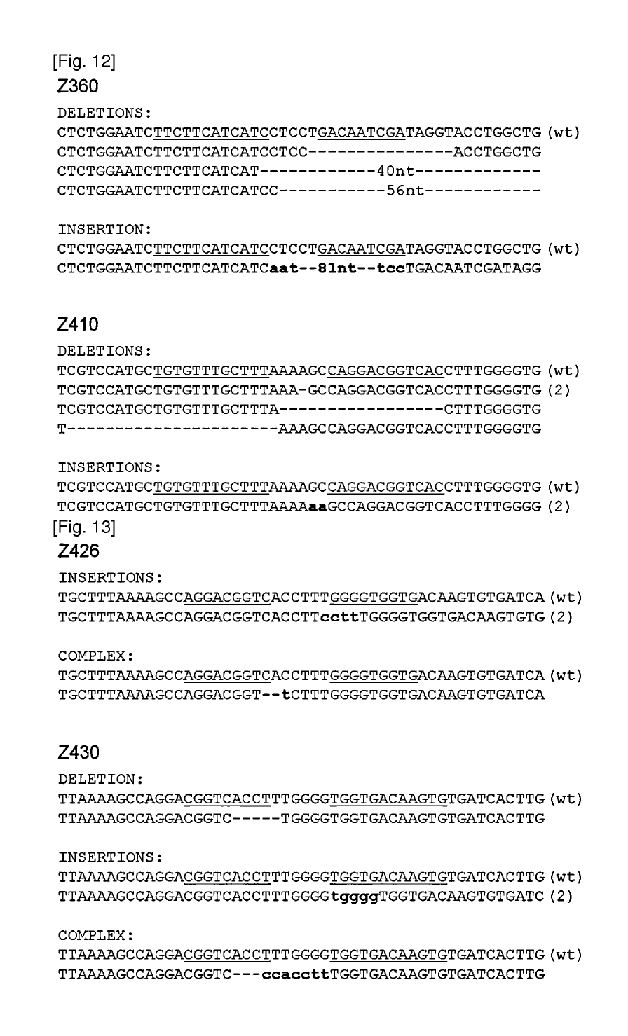

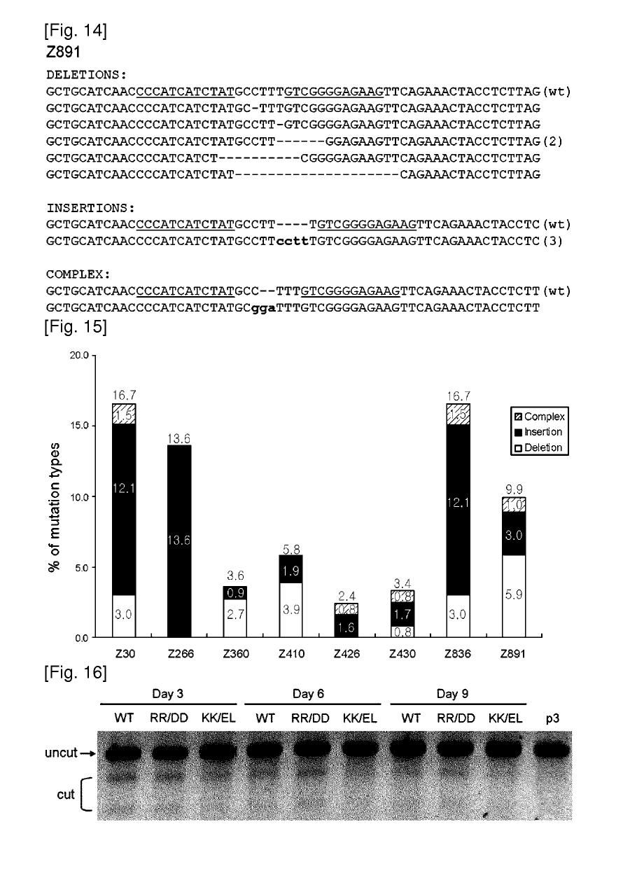

The present inventors then determined the DNA sequences of targeted region to confirm the mutations induced by ZFNs and to estimate the mutagenic rates. Amplified DNA from cells transfected with each of the 8 ZFN pairs was cloned and sequenced. Various deletions and insertions were observed near the sites of DSBs (see FIGS. 10 to 14). These mutagenic patterns are the signature of error-prone NHEJ-associated mutagenesis, and similar patterns have been reported by others (Bibikova, M., Golic, M., Golic, K. G. & Carroll, D. Targeted chromosomal cleavage and mutagenesis in Drosophila using zinc-finger nucleases. Genetics 161, 1169-1175 (2002); Morton, J., Davis, M. W., Jorgensen, E. M. & Carroll, D. Induction and repair of zinc-finger nuclease-targeted double-strand breaks in Caenorhabditis elegans somatic cells. Proc Natl Acad Sci USA 103, 16370-16375 (2006); Santiago, Y. et al. Targeted gene knockout in mammalian cells by using engineered zinc-finger nucleases. Proc Natl Acad Sci USA 105, 5809-5814 (2008)). In addition to the small indel mutations and substitutions, the present inventors observed relatively large insertions (two different ones of 81 nucleotides each) with two ZFN pairs (see Z30 in FIGS. 11 and Z360 in FIG. 12). DNA sequence analysis revealed that the inserted sequences were from the plasmid that encoded the ZFNs.

Eight ZFN pairs that targeted distinct sites showed 2.4% to 17% mutagenic rates (see FIG. 15), which were calculated by dividing the number of mutant clones with the number of total clones analyzed. These high efficiencies of ZFN-induced mutagenesis suggest that clonal mutant cells can be isolated by screening only 10 to 100 single-cell derived colonies.

### EXAMPLE 5

### Isolation of Clonal Cells After ZFN Treatment

5-1. Off-Target Effects of ZFNs of the Present Invention

It has been shown by others that certain ZFNs have off-target effects and are cytotoxic when expressed in mammalian cells (Szczepek, M. et al. Structure-based redesign of the dimerization interface reduces the toxicity of zinc-finger nucleases. Nat Biotechnol 25, 786-793 (2007); Miller, J. C. et al. An improved zinc-finger nuclease architecture for highly specific genome editing. Nat Biotechnol 25, 778-785 (2007)). The present inventors observed no significant growth retardation of HEK293 cells transfected with ZFNs of the present invention. However, when they initially screened a limited number of single-cell colonies, they were unable to isolate clonal mutant cells whose genome sequences were modified by ZFN treatment, which indicates that mammalian cells carrying ZFN-induced mutations were growth-impaired.

Therefore, in order to investigate whether mammalian cells that carry ZFN-induced mutations were growth-impaired and thus outgrown by unmodified cells, the present inventors performed the T7E1 assay with DNA isolated from cells at 3, 6 and 9 days after ZFN transfection. They chose the Z891 pair for this analysis and, as shown in FIG. 16, the cleaved DNA fragments were significantly reduced at day 6, compared with those at day 3, and were barely detectable at day 9 post-ZFN treatment. These results suggest that ZFN-mediated mutagenesis does indeed have cytotoxic effects on cell growth.

5-2. Off-Target effects of Heterodimeric ZFNs of the Present Invention

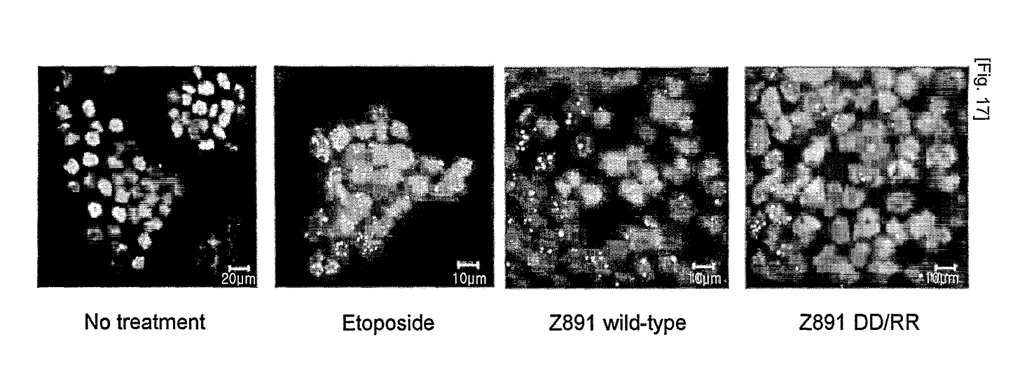

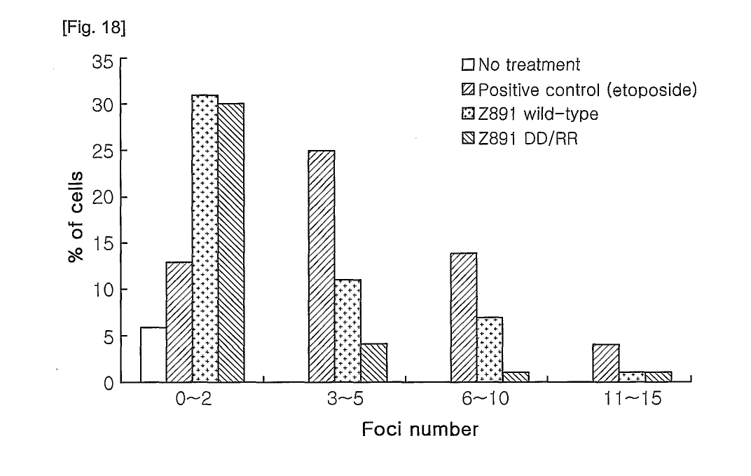

Off-target effects of ZFNs are caused largely by the activity of ZFN monomers that form both homo- and hetero-dimers. These effects could be reduced significantly by using FokI nuclease variants that form hetero-dimers, but cannot form homo-dimers. Therefore, to reduce the cytotoxic effects of ZFNs, the present inventors prepared and tested, in the T7E1 assay, two different types of these so-called obligatory heterodimers (shown as “RR/DD” dimers and “KK/EL” dimers in FIG. 16). The T7E1 assay revealed that, when cells were transfected with the RR/DD ZFN pair, the cleaved DNA fragments persisted even at 9 days after ZFN treatment. In order to confirm that the reduced cytotoxicity of the obligatory heterodimeric ZFN resulted from its enhanced specificity of DNA cleavage in cells, the present inventors analyzed the number of DSBs in ZFN-treated cells using an antibody against 53BP1, a protein that is recruited to DSBs. Specifically, intranuclear stain for 53BP1 was performed with HEK293T/17 cells. Slides were prepared by attaching the cells using a Lab-Tek chamber slide with a cover (NUNC) and fixing the cells with 3.7% formaldehyde. The cells were then permeabilized by treatment with 0.2% Triton X-100 in phosphate-buffered saline (PBS) at room temperature (RT) for 10 min. Cells were then incubated with anti-53BP1 rabbit polyclonal antibodies (Bethyl Laboratories) in the presence of 5% bovine serum albumin (BSA) to block nonspecific staining, followed by incubation with Alexa Fluor 488-conjugated secondary antibodies (Invitrogen-Molecular Probes). Slides were mounted in the presence of DAPI (Sigma) to counterstain cell nuclei and examined under an immunofluorescence microscope (Carl Zeiss-LSM510). The results are shown in FIGS. 17 and 18. As shown in FIGS. 17 and 18, the number of 53BP1 foci was reduced significantly in cells treated with the RR/DD ZFN pair, when compared with cells treated with the corresponding wild-type ZFN pair.

5-3. Mutation Detection in Genome Treated with ZFNs

Next, seven different mutant clonal HEK293 cells were subsequently isolated using the Z891 RR/DD ZFN pair after screening 225 single-cell colonies that had been grown separately in 96-well plates. The present inventors examined the DNA sequences of the CCR5 regions in these clonal cells to confirm the ZFN-induced genomic modifications. DNA sequence analyses of the genomes from the modified cells revealed that 6 of the 7 clones showed monoallelic deletions or insertions and one showed biallelic modifications in the CCR5 gene (see FIG. 19). Because of a partial trisomy, HEK293 cells carry three copies of the CCR5 gene in their genome. In the clone that showed biallelic modifications, one unmodified, wild-type sequence was observed (see FIG. 19). This bialleic modification by ZFN treatment suggests that homozygous knockout cells could be isolated in a single step without the use of selection markers.

### EXAMPLE 6

### Off-Target Effects of CCR5-Targeting ZFNs at the Homologous CCR2 Sites

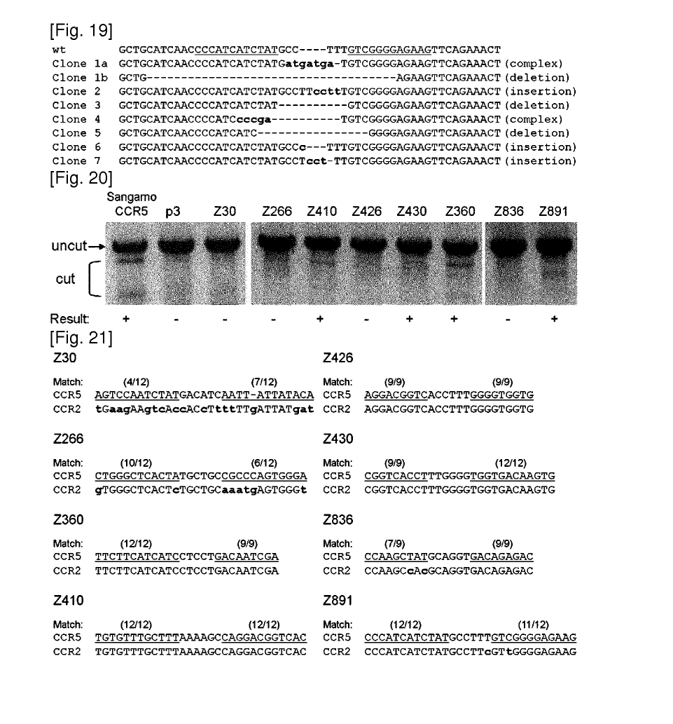

The CCR2 gene is highly homologous to the CCR5 gene, and many of the CCR5 ZFN recognition elements are conserved in the CCR2 locus. Therefore, the present inventors examined whether the 8 ZFNs designed for the CCR5 sites could also modify homologous or identical sequences at the CCR2 locus. Three ZFN pairs (Z360, Z410 and Z430) that have identical recognition elements in the CCR2 gene were able to induce efficient genomic modification in the T7E1 assay (see FIG. 20). In the CCR2 gene, the recognition element of the Z891 pair consisted of a 12-bp half-site that perfectly matched the corresponding site in the CCR5 gene and a 12-bp half-site that carried a one-base mismatch. As expected, this Z891 pair also showed significant gene targeting activity at the CCR2 site. The present inventors also tested Sangamo's CCR5-targeting ZFN pair whose recognition element in the CCR2 locus contains one-base mismatches in each of the 12-bp half-sites and confirmed that this ZFN shows off-target genomic modification at the homologous CCR2 site (see FIG. 20). In contrast, no detectable ZFN activities were observed with ZFN pairs (Z30, Z266 and Z836) whose recognition elements at the CCR2 locus displayed at least two mismatches.

The present inventors also examined whether the 7 mutant clonal cells obtained by transfection of HEK293 cells with the Z891 pair induced mutations in the DNA sequence of the highly homologous CCR2 site in addition to that of the intended CCR5 site. The results are shown in FIG. 21. Although the Z891 pair showed significant gene targeting activity at the CCR2 site as well as at the CCR5 site in the T7E1 assay as described in FIG. 20, the CCR2 locus was not mutated in these clonal cell lines. These results demonstrate that it is possible to isolate clonal cells in which only the intended target site, but not homologous sites, is mutated by screening cells after ZFN treatment.

### EXAMPLE 7

### Module Swap Analysis

The present inventors next investigated why our approach of making ZFNs via modular assembly led to high success rates in genome editing. Specifically, they performed module swap experiments in which the ZFNs of the present invention were replaced with those generated from Barbas or Sangamo modules that displayed identical DNA-binding specificities.

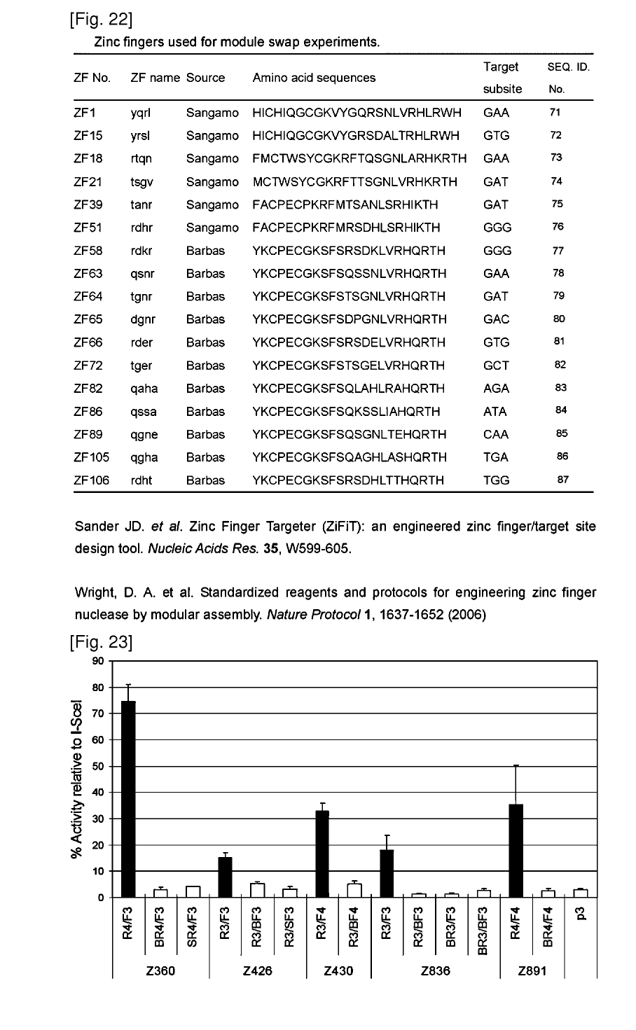

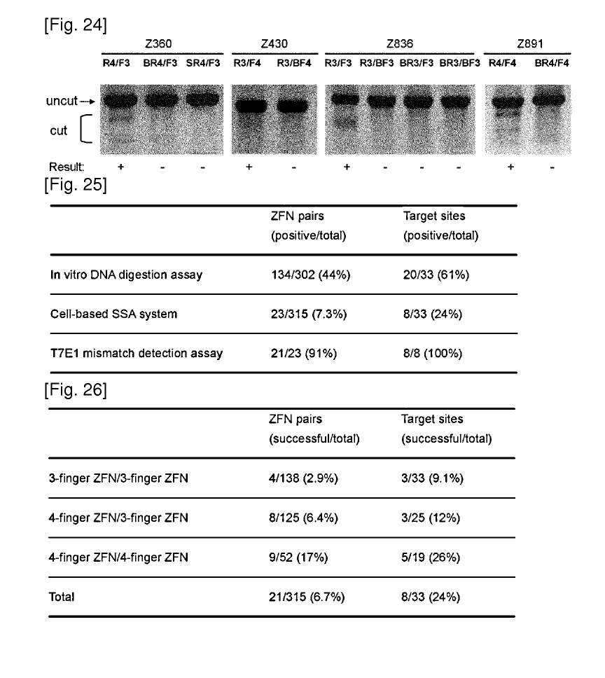

To this end, the present inventors prepared 8 new ZFN monomers composed exclusively of Barbas or Sangamo modules and used them to replace 6 different ZFN monomers composed exclusively of ToolGen modules (see Table 3 and FIG. 22). Each of these new ZFN monomers was paired with appropriate partner ZFN monomers, and the resulting ZFN pairs were tested in both the SSA and T7E1 assays. As shown in FIGS. 23 and 24, none of the ZFN pairs that consisted of at least one of these newly synthesized ZFN monomers showed any significant activity in the SSA system (see FIG. 23). In addition, none of these ZFNs showed any gene targeting activity in the T7E1 assay (see FIG. 24).

TABLE 3
[Table 3]

| | | | | | | Half-site |
|---|---|---|---|---|---|---|
| | | | | | | sequence |
| ZFN | name | F1 | F2 | F3 | F4 | (from 5′ to 3′) |
| Z360 | R4 | QSNR1 | QSNR1 | ISNR | ISNR | GAT GAT GAA GAA |
| | BR4 | ZF63 | ZF63 | ZF64 | ZF64 | GAT GAT GAA GAA |
| | SR4 | ZF1 | ZF18 | ZF21 | ZF39 | GAT GAT GAA GAA |
| | F3 | QSHV | VSNV | DSNR | | GAC AAT CGA |
| Z426 | R3 | tnse | srta | DSNR | | GAC CGT CCT |
| | F3 | RDER2 | RDER1 | RDHR1 | | GGG GTG GTG |
| | BF3 | ZF66 | ZF66 | ZF58 | | GGG GTG GTG |
| | SF3 | ZF15 | ZF15 | ZF51 | | GGG GTG GTG |
| Z430 | R3 | rdte | QSHV | RDKR | | AGG TGA CCG |
| | F4 | RDER2 | QSNV2 | QSHV | RDHT | TGG TGA CAA GTG |
| | BF4 | ZF66 | ZF89 | ZF105 | ZF106 | TGG TGA CAA GTG |
| Z836 | R3 | RDHT | VSTR | QNTQ | | ATA GCT TGG |
| | BR3 | ZF106 | ZF72 | ZF86 | | ATA GCT TGG |
| | F3 | CSNR1 | QTHQ | DSNR | | GAC AGA GAC |
| | BF3 | ZF65 | ZF82 | ZF65 | | GAC AGA GAC |
| Z891 | R4 | RSHR | ISNR | ISNR | QNTQ | ATA GAT GAT GGG |
| | BR4 | ZF58 | ZF64 | ZF64 | ZF86 | ATA GAT GAT GGG |
| Z891 | F4 | rdnq | QFNR | RSHR | DSAR2 | GTC GGG GAG AAG |

These results strongly support the proposal that naturally-occurring ZFs constitute a more reliable framework for the modular assembly of functional ZF arrays than do engineered ZFs.

### EXAMPLE 8

### Evaluating ZF Modules of the Present Invention

Statistical analysis of 315 ZFN pairs that were produced by the present inventors could provide a basis for the design of new ZFNs that can be used for targeted mutagenesis of additional endogenous genes of interest. To this end, the present inventors counted the number of occurrences of each ZF in the 23 ZFN pairs that scored positively in the SSA system. They then determined an “activity score” for each ZF by dividing this number with the number of occurrences of the module in all 208 ZFN monomers (see FIGS. 1 and 2). Certain highly active ZFs, for example, “QNTQ” and “RSHR”, had activity scores of 50% and 38%, respectively; these high activity scores suggest that these modules are reliable when it comes to predicting in vivo site-specific nuclease activity. Other ZFs, such as “QSNK” and “rdae”, which were shown to be inefficient modules in the present assays, displayed activity scores of zero. Both of these ZFs were used 15 times each, but none of the ZFNs that contained these modules were active in our assays. The present inventors also compared the activity scores of ZFs with identical DNA recognition specificities. Certain ZFs were clearly better than other target-equivalent modules. For example, both “DSNR” and “HSNK” recognized the same GAC sequence. The use of “DSNR” in the construction of ZFNs yielded many active nucleases (activity score =35%), while the incorporation of “HSNK” yielded only inactive ZFNs (activity score =0%).

On the basis of the statistical analysis, the present inventors tentatively recommend 37 ZFs (24 ToolGen modules, 1 Sangamo module, and 12 Barbas modules) for use in future gene editing studies (see FIGS. 1 and 2). If the present inventors had used only these 37 modules in the experiments, the overall success rate would have been 53%, which is a significant improvement over the current rate (24%) (see FIG. 26). These 37 modules all were found at least once in active ZFNs. When there were two or more target-equivalent ZFs, they selected those with the higher activity scores. The 37 ZFs collectively recognize 38 out of 64 3-bp sub-sites. For a given 1-kbp random DNA sequence, they predict that there would be 88 [=(38/64)⁶×1000×2] potential target sites (that contain either a 5- or 6-base spacer between the half sites) for 3-finger ZFN dimers and 31 sites for 4-finger ZFN dimers. Assuming 9.1% to 26% success rates by the present invention (the actual rates would be significantly higher if we use the 37 ZFs, but not the other inefficient modules), ZFN-mediated genome editing would be possible at 8 sites, on average, in the 1-kbp target sequence. This suggests that most, if not all, eukaryotic genes should be “targetable” with the ZFN assembly method of the present invention.

### INDUSTRIAL APPLICABILITY

In many gene therapy applications, these zinc finger nucleases might be used for knocking out CCR5 gene to produce T cells that are resistant to HIV infection in AIDS patients.

1. A fusion protein having nuclease activity, comprising a zinc finger domain and a nucleotide cleavage domain, wherein the zinc finger domain is engineered by assembling three or more zinc finger modules for binding with a nucleotide sequence, and
at least one of the zinc finger modules are from the naturally-occurring, wild-type zinc finger modules.

2. The fusion protein according to claim 1, wherein the zinc finger domain comprises three or four zinc finger modules.

3. The fusion protein according to claim 1, wherein the zinc finger module is any one of the modules that are described in FIG. 1.

4. The fusion protein according to claim 1, wherein the fusion protein functions as a dimer to cleave a nucleotide sequence.

5. The fusion protein according to claim 4, wherein the fusion protein is any one of the proteins that are described in Table 2.

6. The fusion protein according to claim 1, wherein the nucleotide cleavage domain is the cleavage domain from the type IIS restriction endonuclease.

7. (canceled)

8. A kit for cleavage, replacement or modification of nucleotide sequences in targeted region, comprising one or more pair of fusion proteins as in claim 1.

9. The kit according to claim 8, wherein the pair of fusion protein have the same or different zinc finger domains.

10-13. (canceled)

14. A method for producing a the fusion protein having nuclease activity as in claim 1, comprising the steps of:
(a) selecting a nucleotide sequence in the region of interest;
(b) selecting zinc finger modules to bind to the sequence and engineering a zinc finger domain, wherein the zinc finger domain is assembled by three or more zinc finger modules, at least one of the zinc finger modules are from the naturally-occurring, wild-type zinc finger modules; and
(c) expressing the fusion protein that comprises the zinc finger domain and a nucleotide cleavage domain.

15-20. (canceled)

21. A method for cleaving a nucleotide in a region of interest, comprising:
cleaving the nucleotide sequence in the region of interest by the fusion protein as in claim 1.

22. The method according to claim 21,
wherein a first fusion protein and a second fusion protein are used;
the first fusion protein comprising a first zinc finger domain and a first nucleotide cleavage domain;
the second fusion protein comprising a second zinc finger domain and a second nucleotide cleavage domain; and
when the first fusion protein binds a first nucleotide sequence, the second fusion protein is bound to a second nucleotide sequence which is located between 2 and 50 nucleotides from the first nucleotide sequence.

23. The method according to claim 22, wherein the cleavage is performed between the first and second nucleotide sequences.

24-31. (canceled)

32. The method according to claim 22, wherein the first and second nucleotide cleavage domains are from the same endonuclease.

33-34. (canceled)

35. A method for replacing a first nucleotide sequence in a region of interest comprising:
(a) cleaving the first nucleotide sequence with a first fusion protein as in claim 1 and a second fusion protein as claim 1;
the first fusion protein comprising a first zinc finger domain and a first nucleotide cleavage domain; the first zinc finger domain being engineered for binding to a second nucleotide sequence having at least 9 nucleotides; and
the second fusion protein comprising a second zinc finger domain and a second nucleotide cleavage domain; the second zinc finger domain being engineered for binding to a third nucleotide sequence having at least 9 nucleotides which is located between 2 and 50 nucleotides from the second nucleotide sequence; and
(b) adding a polynucleotide comprising a fourth nucleotide sequence non-identical with the first nucleotide sequence;
wherein binding of the first fusion protein to the second nucleotide sequence; and binding of the second fusion protein to the third nucleotide sequence lead to cleavage in the region of interest,
thereby facilitating a homologous recombination between the first nucleotide sequence and the fourth nucleotide sequence, resulting in replacement of the first nucleotide sequence with the fourth nucleotide sequence.

36-45. (canceled)

46. A method for altering a first nucleotide sequence in a region of interest comprising:
cleaving the first nucleotide sequence with a first fusion protein as in claim 1 and a second fusion protein as in claim 1;
wherein the first fusion protein comprises a first zinc finger domain and a first nucleotide cleavage domain; the first zinc finger domain being engineered for binding to a second nucleotide sequence having at least 9 nucleotides; and
the second fusion protein comprises a second zinc finger domain and a second nucleotide cleavage domain; the second zinc finger domain being engineered for binding to a third nucleotide sequence having at least 9 nucleotides which is located between 2 and 50 nucleotides from the second nucleotide sequence; and
binding the first fusion protein to the second nucleotide sequence; and binding the second fusion protein to the third nucleotide sequence, lead to the cleavage in the region of interest, resulting in the alteration of the first nucleotide in the cleavage site.

47-52. (canceled)

53. A nucleotide encoding the fusion protein as in claim 1.
